SEQUENCE LISTING (14 sequences)

SEQ ID NO: 1 - protein, 5 aa, Artificial Sequence
  Peptide
CALNN

SEQ ID NO: 2 - protein, 10 aa, Artificial Sequence
  Peptide
  MISC_FEATURE (6)..(6): X can be any natural or non-natural aromatic amino acid
CALNNXGGGG

SEQ ID NO: 3 - protein, 15 aa, Artificial Sequence
  trypsin substrate
CALNNYGGGGVRGNF

SEQ ID NO: 4 - protein, 16 aa, Artificial Sequence
  endoproteinase Glu-C substrate
CALNNYGGGGNNESWH

SEQ ID NO: 5 - protein, 12 aa, Artificial Sequence
  Peptide
CALNNYGGGGVR

SEQ ID NO: 6 - protein, 15 aa, Artificial Sequence
  Peptide
  MISC_FEATURE (6)..(6): X can be Y, W or F or any non-natural aromatic amino acid
  MISC_FEATURE (15)..(15): X can be Y, W or F or any non-natural aromatic amino acid
CALNNXGGGGVRGNX

SEQ ID NO: 7 - protein, 15 aa, Artificial Sequence
  Peptide
  MISC_FEATURE (5)..(5): X can be Y, W or F or any non-natural amino acid
  MISC_FEATURE (14)..(14): X can be Y, W or F or any non-natural amino acid
CALNXGGGGNNESXH

SEQ ID NO: 8 - protein, 6 aa, Artificial Sequence
  Peptide
CALNNY

SEQ ID NO: 9 - protein, 4 aa, Artificial Sequence
  Peptide
GGGG

SEQ ID NO: 10 - protein, 6 aa, Artificial Sequence
  Peptide
CCALNN

SEQ ID NO: 11 - protein, 15 aa, Artificial Sequence
  Peptide
  MISC_FEATURE (6)..(6): X is non-natural aromatic amino acid cnF.
CALNNXGGGGVRGNF

SEQ ID NO: 12 - protein, 15 aa, Artificial Sequence
  Peptide
  MISC_FEATURE (6)..(6): X is non-natural aromatic amino acid bzF.
CALNNXGGGGVRGNF

SEQ ID NO: 13 - protein, 4 aa, Artificial Sequence
  recognition site
YVAD

SEQ ID NO: 14 - protein, 6 aa, Artificial Sequence
  Peptide
CCALNN

(19) United States
(12) Patent Application Publication
(10) Pub. No.: US 2021/0079444 A1
(43) Pub. Date: Mar. 18, 2021

(54) LABEL FREE DETECTION OF PROTEASE ACTIVITY

(71) Applicants: VIB VZW, Gent (BE); UNIVERSITEIT GENT, Gent (BE); IMEC VZW, Leuven (BE)

(72) Inventors: Mohamed Lamkanfi, Overmere (BE); Kris Gevaert, Zele (BE); Pieter Wuytens, Gent (BE); Roel Baets, Gent (BE)

(21) Appl. No.: 16/627,229

(22) PCT Filed: Jun. 27, 2018
(86) PCT No.: PCT/EP2018/067311

§ 371 Date: Dec. 27, 2019

(30) Foreign Application Priority Data
Jun. 29, 2017 (GB) 1710401.9

(51) Int. Cl. C12Q 1/37; G01N 21/65

(52) U.S. Cl. CPC C12Q 1/37; G01N 21/65

(57) ABSTRACT

The present disclosure relates to the field of biochemistry, more particularly to detecting and measuring enzyme activity, even more particularly to detecting and measuring protease or nuclease activity. The means and methods disclosed in this application make use of Raman scattering, more particularly of surface enhanced Raman scattering (SERS). The disclosure discloses a carrier on which a monolayer of sequences containing a specific cleavage site is bound. The sequences comprise at least 2 Raman scatterers, one before and one behind the cleavage site, thereby providing an inherent control against ligand exchange.

### CROSS-REFERENCE TO RELATED APPLICATIONS

This application is a national phase entry under 35 U.S.C. § 371 of International Patent Application PCT/EP2018/067311, filed Jun. 27, 2018, designating the United States of America and published as International Patent Publication WO 2019/002401 A1 on Jan. 3, 2019, which claims the benefit under Article 8 of the Patent Cooperation Treaty to Great Britain Patent Application Serial No. 1710401.9, filed Jun. 29, 2017.

### TECHNICAL FIELD

The present disclosure relates to the field of biochemistry, more particularly to detecting and measuring enzyme activity, even more particularly to detecting and measuring protease or nuclease activity. The means and methods disclosed in this application make use of Raman scattering, more particularly of surface enhanced Raman scattering (SERS). The disclosure shows a carrier on which a monolayer of sequences comprising an enzyme cleavage site is bound. The sequences comprise at least 2 Raman scatterers, one before and one behind the cleavage site, thereby providing an inherent control against ligand exchange.

### BACKGROUND

Proteases are enzymes catalysing the hydrolysis of peptide bonds and play a crucial role in the modification of proteins as well as in the breakdown into their constituent amino acids (protein catabolism). Proteases are also of vital importance in numerous signaling pathways (ref 1). A sensitive and quantitative analysis of protease activity is thus of critical importance for, amongst others, medical diagnostics (ref 2), drug development (refs 1-3) and single cell analysis (refs 4,5). As over 500 different genes encoding for proteases have been identified in the human genome, there is large interest in a detection technology that allows for a selective, sensitive and multiplexed measurement of protease activity. Currently established methods such as colorimetric or fluorescent assays and liquid chromatography lack sensitivity or are not scalable to real-time, multiplexed assays. In recent years, several strategies based on nanoparticle-peptide complexes (refs 6-8) have been developed for detecting protease activities. These are typically based on fluorescence-quenching or -energy transfer, offering high sensitivity in in-vitro and even in-vivo measurements. However, fluorescence-based methods provide limited multiplexing capabilities. In contrast, the specificity of Raman fingerprints enables spectrally multiplexed measurements (ref 9). Furthermore, Surface-enhanced Raman scattering (SERS) provides a promising technology for sensitive and selective detection of peptide bond hydrolysis because the nanometer-sized peptides (to be used as substrates) match well with the typical size of a plasmonic hot-spot. Not surprisingly, a number of studies reported on detecting protease activity based on SERS (refs 10-14) and on surface-enhanced resonance Raman scattering (SERRS) (refs 15-17). These include indirect sensing methods based on clustering (ref 14) and anti-clustering (ref 13) of nanoparticles upon cleavage, and direct detection methods based on the appearance (ref 17) and disappearance (refs 10,11) from the spectra of SERS labels. These studies demonstrate the ability for detecting protease activities at single-cell concentrations levels in sub-nanoliter volumes (refs 11,13,15). Some of these findings are also subject of patent applications, e.g., WO2009094058 and WO2008018933A3.

In this application, a real-time detection of protease-mediated peptide bond hydrolysis through the SERS spectra of surface-bound peptides consisting of only natural or non-natural amino acids is disclosed. The inherent SERS fingerprint of aromatic amino acids (refs18,19) is used in the peptides, which eliminates the use of fluorescent- or SERS-labels. As a consequence, the peptide design is easily adjustable toward a specific substrate for different proteases. These peptides form a self-assembled monolayer (ref 20) on a gold-nanodome patterned SERS platform (ref 21), with distinguishable vibrational spectra both before and after the cleavage site. This enables discriminating specific protease cleavage from ligand exchange (refs 22,23). A stepwise approach is taken to prove that the changes in SERS spectra upon peptide hydrolysis indeed originate from substrate cleavage. First, Raman spectra of the pure peptide are compared to SERS spectra acquired on the nanodome surface. Peaks are attributed to known vibrations in literature and to own experimental data. Investigated next is the SERS spectra of the cleaved products separated by reverse phase high-performance liquid chromatography (RP-HPLC) after incubating specific substrates with the serine proteases trypsin and endoproteinase Glu-C. It is also verified that the peptides are bound to the nanodome gold surface through an amine-terminal cysteine, correctly presenting the cleavage site away from the solid interface. Finally, there is a demonstration of the digestion of a surface-bound trypsin substrate and this reaction is followed in real-time through continuous changes in the SERS spectra.

### BRIEF SUMMARY

Surface-enhanced Raman scattering provides a promising technology for a sensitive and selective detection of protease activity by monitoring peptide cleavage. Not only are peptides and plasmonic hotspots similarly sized, Raman fingerprints also hold large potential for spectral multiplexing. However, current available methods (e.g., U.S. Pat. No. 8,685,743; US20140011705) do not distinguish between cleavage of the C-terminally attached Raman scatterer and ligand exchange on the surface. This forces the practitioner to duplicate the experiments with protease inhibitors as control. Here, Applicant discloses a gold-nanodome platform for a real-time detection of protease activity with an inherent control against ligand exchange. It is further demonstrated that the relative intensity of the fingerprints from aromatic amino acids before and behind the cleavage site provides a robust figure of merit for the turnover rate, thereby eliminating the need for high mass Raman scatterers such as Rhodamine. The presented method offers a generic approach for measuring protease activity, which is illustrated by developing a substrate for both trypsin and endoproteinase Glu-C, in a multiplexed way compatible with the use of waveguides.

Therefore in a first aspect, the application provides a substrate for protease measurements, wherein the substrate is a peptide with a maximum length of 35 amino acids, the peptide comprising at least 2 Raman active tags, wherein the at least 2 Raman active tags comprise at least 1 Raman active tag N-terminally from a protease recognition sequence and at least 1 Raman active tag C-terminally from the protease recognition sequence. In particular embodiments, the Raman active tag is a tag for surface enhanced Raman scattering (SERS) detection. In other particular embodiments, the Raman active tags consists of natural and/or non-natural aromatic amino acids. In more particular embodiments, the substrate or peptide comprises SEQ ID NO:1 of the SEQUENCE LISTING incorporated herein by this reference: CALNN or comprises an amino acid sequence having 90% homology to SEQ ID NO:2: CALNNXGGGG, wherein X can be any natural or non-natural aromatic amino acid.

The application envisions detection of enzymatic activity and/or presence of an enzyme using a carrier such as the gold-nanodome platform as demonstrated in the Examples of this application. Therefore in a second aspect, a carrier having a molecule attached to it is provided, the molecule comprises an enzyme recognition sequence and at least 2 Raman active tags, wherein the at least 2 Raman active tags comprise at least 1 Raman active tag before the enzyme recognition sequence and at least 1 Raman active tag behind the enzyme recognition sequence. Besides detecting or quantifying the presence, amount or activity of an enzyme, multiple enzymes can be detected or quantified at one time in one sample. Therefore, a carrier is provided having n different molecules attached to it, wherein the n different molecules differ from each other by comprising a different or an unique enzyme recognition sequence, wherein every molecule comprises at least one Raman active tag before and at least one Raman active tag behind the enzyme recognition sequence, wherein the ratios of the Raman intensity of the at least one Raman tag behind the enzyme recognition sequence and the Raman intensity of the at least one Raman tag before the enzyme recognition sequence is specific for every molecule comprising a different or an unique enzyme recognition sequence, and wherein n is an integer between 2 and 100. For either single or multiplex uses, the molecule or molecules attached to the carrier can be a nucleic acid sequence or nucleic acid sequences and consequently the enzyme is then a nuclease. The application also provides a carrier where the molecule or molecules attached to it are peptides or amino acid sequences and hence the enzyme is then a protease. In particular embodiments, a carrier is provided where the peptide or amino acid sequence attached to it is any of the peptides disclosed in this application. In more particular embodiments, the carrier comprises a material selected from the list consisting of gold, silver, silicon, silicon nitride, silicon dioxide, quartz, polystyrene, silica and dextran.

The above described peptides and carriers are particularly useful for the detection or quantification of the presence, amount or activity of one or more enzymes in a biological sample. Given that many diseases are associated with an altered activity or presence of one or more enzymes, the carriers from current application are disclosed for use as diagnostic.

In another aspect, a method of detecting or quantifying the presence, amount or activity of an enzyme in a biological sample is provided, the method comprising:
a. contacting one of the carriers of the application with the sample, wherein the molecule attached to the carrier comprises a recognition sequence for the enzyme;
b. measuring the Raman intensity ratio before and after contacting the sample with the carrier, wherein the Raman intensity ratio is the ratio between the Raman intensity of the one or more Raman active tags behind the enzyme recognition sequence and the Raman intensity of the one or more Raman active tags before the enzyme recognition sequence or vice versa;

wherein a change in Raman ratio before and after contacting the sample with the carrier gives a metric for the presence, amount or activity of the enzyme in the sample. Given that subject-matter of this application is particularly useful for multiplex detection and/or quantification, also a method is provided for the multiplex detection or quantification of n different enzymes in one biological sample, the method comprising:
a. contacting the carrier as described above having n different molecules attached to it with the sample, wherein the n different molecules attached to the carrier comprise the recognition sequences for the n different enzymes;
b. measuring the Raman intensity ratios before and after contacting the sample with the carrier, wherein the Raman intensity ratios are the ratios between the Raman intensity of the one or more Raman active tags behind the enzyme recognition sequence of one of the n different molecules and the Raman intensity of the one or more Raman active tags before the enzyme recognition sequence of the one of the n different molecules or vice versa;

wherein for each different enzyme and for the Raman active tags of a molecule comprising the recognition sequence of the enzyme, a change in Raman ratios before and after contacting the sample with the carrier gives a metric for the presence, amount or activity of the n different enzymes in the sample.

In both described methods, the enzyme can be a protease or a nuclease and accordingly the attached molecules are peptides or nucleic acid sequences, respectively. A particular advantage of developing above described peptides and carriers is that the Raman scattering used can be excited and collected by one or more integrated optical waveguides. In particular embodiments, the one or more integrated optical waveguides are integrated dielectric optical waveguides and patterned with gold, silver, copper or aluminum nanostructures.

The subject-matter of current application can also be used for drug discovery. Therefore and in yet another aspect, a method is provided to screen for compounds with protease or nuclease activity, the method comprising:
a. contacting any of the carriers of current application with at least one test compound;
b. measuring the Raman intensities of the at least two Raman active tags of the one or more different molecules attached to the carrier before and after contacting the test compound with the carrier;

identifying the test compound as a compound with protease or nuclease activity, if at least one Raman ratio decreases with at least 25% after contacting the carrier with the test compound, wherein the Raman ratio is the ratio between the Raman intensity of the one or more Raman active tags behind an enzyme recognition sequence of a molecule attached to the carrier and the Raman intensity of the one or more Raman active tags before the enzyme recognition sequence.

### BRIEF DESCRIPTION OF THE DRAWINGS

FIG. 1. (a) A gold-bound peptide substrate provides a SERS fingerprint with distinctive peaks from amino acids on both sides of the cleavage site (SEQ ID NO:3). After protease hydrolysis, the non-bound product diffuses away and its Raman peaks disappear. (b) The peptide substrate designed for trypsin hydrolysis, which cleaves C-terminal to arginine (R) residues.

FIG. 2. (a) Schematic and (b) corresponding tilted SEM figures of the gold nanodome patterned microchip fabrication. A monolayer of polystyrene beads (1-2) forms a mask for reactive ion etching into the underlying Si3N4 (3-4), resulting in an array of nanopillars that serve as template for gold deposition (5).

FIG. 3. (a) Top-down and cross-section SEM images for a nanodome-pattern chip with a 12 nm wide (g) and 53 nm high (h) inter-dome gap. (b) Corresponding |E|2 field distributions for this geometry at resonance wavelength simulated through 3D FDTD, showing that the localized surface plasmonic resonance is confined to the inter-dome gap region. The field distributions are plotted over the region corresponding to the yellow rectangles.

FIG. 4. (a1) Raman and SERS spectra of the CALNNYGGGGVRGNF (SEQ ID NO:3) trypsin substrate show characteristic tyrosine and phenylalanine peaks. (a2) RP-HPLC was performed after incubation of this peptide with trypsin and eluting peaks were analyzed by mass spectrometry, which shows almost full digestion after 30 minutes of incubation with trypsin. (a3) SERS spectra of RP-HPLC separated fractions and their difference spectra (a4) confirm the full disappearance of F-related peaks upon trypsin digestion. (b1) Raman and SERS spectra of the CALNNYGGGGNNESWH (SEQ ID NO:4) endoproteinase Glu-C substrate with characteristic tryptophan peaks. (b2) RP-HPLC and mass spectrometry analysis after incubation with endoproteinase Glu-C show an almost complete digestion after 4 hours of incubation. (b3-b4) SERS spectra and difference spectrum of the RP-HPLC separated fractions show the disappearance of tryptophan-related peaks after cleavage.

FIG. 5. (a) SERS spectra of a peptide solution before (red) and after (green) trypsin incubation prove that the binding of the peptide (SEQ ID NO:3) to the gold surface happens via the side-chain of the N-terminal cysteine through a gold-sulphur bond, but not via the free amine part of the −GNF product. (b) The difference spectrum before and after cleavage agrees with the difference between the RP-HPLC separated uncleaved and cleaved fractions shown in FIG. 4.

FIG. 6. Trypsin cleavage of surface-bound CALNNYGGGGVRGNF (SEQ ID NO:3) peptides. (a) SERS spectra with the characteristic tyrosine and phenylalanine peaks highlighted in blue and pink. (b) Relative intensity of the highlighted peaks (IF/IY), showing peptide cleavage in the presence of trypsin, which is blocked when an ovomucoid (Type II-0) inhibitor is added. (c) Difference spectrum between the sample with and without trypsin, which agrees well with the results of the bulk digestion in FIG. 4 and FIG. 5.

FIG. 7. Real-time trypsin digestion of gold-nanodome bound peptides. (a) Evolution of SERS spectra before and after trypsin addition, scaled for equal 1003 cm-1 intensity. This shows a relative increase of CALNNYGGGGVR (SEQ ID NO:5)-related peaks at 829, 860, 948, 1248, 1330, 1603 and 1677 cm-1 versus the 1003 cm-1 phenylalanine peak upon trypsin addition (t=0). (b) SERS spectra at individual time points, the inset zooms in on the 1003 cm-1 F-peak. (c) Time evolution of IF/IY, characterized by 11003/1829-860 and measured in four different experiments, all showing a fast cleavage within the first minutes after trypsin addition.

FIG. 8. Illustration of a multiplexed detection of two peptides.

FIG. 9. Nanotriangle-patterned silicon-nitride waveguide.

FIG. 10. Detection of surface-enhanced Raman signal of peptide monolayer through the waveguide (top) versus microscope-based measurements on nanotriangles and nanodomes. The bottom curve is the spontaneous Raman spectrum of the peptide (SEQ ID NO:15. Gray lines highlight the most prominent peaks of F and pNA. (Spectra are cascaded and scaled for clarity).

FIG. 11. Top view of a possible chip-layout of a waveguide-based, 4-channel SERS measurement. Each channel provides a multiplexed detection of the activity of two proteases (P1 and P2).

FIG. 12. Schematic of a hybrid Si₃N₄—Al₂O₃—Au waveguide. The left inset shows the propagating plasmon mode excited using the fundamental TE mode of the dielectric access waveguide. Stokes power P_{S} is collected in back-reflection. The figures on the right are scanning-electron microscopy images of a typical device in top view (top) and cross-section (bottom).

FIG. 13. Raman spectra of Trypsin substrates incorporating natural and non-natural aromatic amino acids (from top to bottom, SEQ ID NO:3, SEQ ID NO:12, and SEQ ID NO:11, respectively). On the right, the chemical structures are shown of the aromatic amino acids that, respectively, stay on (left) and get cleaved off (right) the surface. An exemplary specific peak for the different aromatics is shaded in the spectra.

FIG. 14. Trypsin digestion on the different designed peptides, observed from Raman spectra before and after bulk cleavage. In all three measurements, a decrease in the 1003 cm⁻¹ Phenylalanine peak is observed from the difference spectra. Pink shaded areas are exemplary peaks of the aromatic amino acid remaining on the gold surface after cleavage. (a1-a2) CALNNYGGGGVRGNF (SEQ ID NO:3), with containing Tyr (833-853 cm⁻¹) and Phe (1003 cm⁻¹) peaks highlighted. (b1-b2) CALNN(cnF)GGGGVRGNF (SEQ ID NO:11) peptide, cyano-Phe (1180 cm⁻¹) and Phe peaks highlighted. (c1-c2) CALNN(bzF)GGGGVRGNF (SEQ ID NO:12) peptide, benzoyl-Phe (1151 cm⁻¹ and 1003 cm⁻¹) peaks highlighted. Note that this last peptide has a double contribution from Phe and benzoyl-Phe at 1003 cm⁻¹, hence the peak does not disappear completely after cleavage.

### DETAILED DESCRIPTION

### Definitions

The present disclosure will be described with respect to particular embodiments and with reference to certain drawings but the disclosure is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g., “a” or “an”, “the”, this includes a plural of that noun unless something else is specifically stated.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the disclosure described herein are capable of operation in other sequences than described or illustrated herein.

The following terms or definitions are provided solely to aid in the understanding of the disclosure. Unless specifically defined herein, all terms used herein have the same meaning as they would to one skilled in the art of the present disclosure. Practitioners are particularly directed to Michael R. Green and Joseph Sambrook, Molecular Cloning: A Laboratory Manual, 4^{th} ed., Cold Spring Harbor Laboratory Press, Plainsview, N.Y. (2012); and Ausubel et al., Current Protocols in Molecular Biology (Supplement 47), John Wiley & Sons, New York (1999), for definitions and terms of the art. The definitions provided herein should not be construed to have a scope less than understood by a person of ordinary skill in the art.

In current application, means and methods are provided for the detection of enzymatic activity using Surface Enhance Raman Scattering or SERS. Raman scattering, also known as the Raman effect, is the inelastic scattering of a photon by molecules, which are excited to higher vibrational or rotational energy levels (Zhang et al. 2011 Curr Pharm Biotechnol 11:654-661). In inelastic (Raman) scattering, an absorbed photon is re-emitted with lower energy; the difference in energy between the incident photons and scattered photons corresponds to the energy required to excite a molecule to a higher vibrational mode. Typically, in Raman spectroscopy high intensity laser radiation with wavelengths in either the visible or near-infrared regions of the spectrum is passed through a sample. Surface-Enhanced Raman Scattering is a technique that enhances Raman scattering by molecules absorbed on rough metal surfaces.

In a first aspect, a peptide is provided with a maximum length of 35 amino acids, wherein the peptide comprises a protease recognition sequence and at least 2 Raman active tags, wherein the at least 2 Raman active tags comprise or are at least 1 Raman active tag N-terminally from the protease recognition sequence and at least 1 Raman active tag C-terminally from the protease recognition sequence. In a particular embodiment, the protease recognition sequence is at least one protease recognition sequence.

Said peptide is designed for the detection of SERS signals. SERS stands for Surface Enhance Raman Scattering, wherein the peptide is attached to the surface. The peptide is attached to the surface with its N-terminus or with its C-terminus. The total length of the peptide must remain limited to 5 nanometers because of decreasing SERS signal with increasing distance from the C-terminus of the peptide to the surface. Therefore, in one embodiment, a peptide is provided with a maximum length of 5 nanometers, wherein the peptide comprises a protease recognition sequence and at least 2 Raman active tags, wherein the at least 2 Raman active tags comprise at least 1 Raman active tag N-terminally from the protease recognition sequence and at least 1 Raman active tag C-terminally from the protease recognition sequence.

A distance of 5 nanometers can be translated to a length of approximately 20 to 30 amino acids. Thus, in one embodiment, the peptide has a maximum length of 30 amino acids, or of 29 amino acids, or of 28 amino acids, or of 27 amino acids, or of 26 amino acids, or of 25 amino acids, or of 24 amino acids, or of 23 amino acids, or of 22 amino acids, or of 21 amino acids, or of 20 amino acids. In another embodiment, the peptide has a length between 14 and 33 amino acids, or between 10 and 25 amino acids or between 12 and 20 amino acids or between 11 and 18 amino acids.

In yet another embodiment, the Raman active tag is a tag for surface enhanced Raman scattering (SERS) detection. The person skilled in the art is familiar with Raman active tags and multiple Raman active tags are available in the art (Wang et al 2012 Chemical Reviews 113: 1391-1428). A Raman active tag generates a specific Raman spectrum by which the presence of the tag can be detected. A molecule (e.g., a non-aromatic amino acid) that generates a generic Raman signal based on the backbone structure of the molecule is not considered as a Raman active tag.

Non-limiting examples of Raman active tags are fluorescent dyes such as Cy3, Cy5, and rhodamine. These tags can be covalently or non-covalently attached to the peptide. In particular embodiments of the first aspect, the Raman active tags are aromatic amino acids, wherein the aromatic amino acids can be natural or non-natural. Non-limiting examples of natural aromatic amino acids are tyrosine, tryptophan or phenylalanine. Non-limiting examples of non-natural aromatic amino acids are: 3-Acetamidobenzoic acid, 4-Acetamidobenzoic acid, 4-Acetamido-2-methylbenzoic acid, N-Acetylanthranilic acid, 3-Aminobenzoic acid, 3-Aminobenzoic acid hydrochloride, 4-Aminobenzoic acid, 4-Aminobenzoic acid potassium salt, 4-Aminobenzoic acid sodium salt, 4-Aminobenzoic acid sodium salt hydrate, 2-Aminobenzophenone-2′-carboxylic acid, 2-Amino-4-bromobenzoic acid, 2-Amino-5-bromobenzoic acid, 3-Amino-2-bromobenzoic acid, 3-Amino-4-bromobenzoic acid, 3-Amino-5-bromobenzoic acid, 4-Amino-3-bromobenzoic acid, 5-Amino-2-bromobenzoic acid, 2-Amino-3-bromo-5-methylbenzoic acid, 2-Amino-3-chlorobenzoic acid, 2-Amino-4-chlorobenzoic acid, 2-Amino-5-chlorobenzoic acid, 2-Amino-6-chlorobenzoic acid, 3-Amino-2-chlorobenzoic acid, 3-Amino-4-chlorobenzoic acid, 4-Amino-2-chlorobenzoic acid, 4-Amino-3-chlorobenzoic acid, 5-Amino-2-chlorobenzoic acid, 4-Amino-5-chloro-2-methoxybenzoic acid, 2-Amino-5-chloro-3-methylbenzoic acid, 3-Amino-2,5-dichlorobenzoic acid, 4-Amino-3,5-dichlorobenzoic acid, 2-Amino-4,5-dimethoxybenzoic acid, 4-(2-Aminoethyl)benzoic acid hydrochloride, 2-Amino-4-fluorobenzoic acid, 2-Amino-5-fluorobenzoic acid, 2-Amino-6-fluorobenzoic acid, 4-Amino-2-fluorobenzoic acid, 2-Amino-5-hydroxybenzoic acid, 3-Amino-4-hydroxybenzoic acid, 4-Amino-3-hydroxybenzoic acid, 2-Amino-5-iodobenzoic acid, 5-Aminoisophthalic acid, 2-Amino-3-methoxybenzoic acid, 2-Amino-4-methoxybenzoic acid, 2-Amino-5-methoxybenzoic acid, 3-Amino-2-methoxybenzoic acid, 3-Amino-4-methoxybenzoic acid, 3-Amino-5-methoxybenzoic acid, 4-Amino-2-methoxybenzoic acid, 4-Amino-3-methoxybenzoic acid, 5-Amino-2-methoxybenzoic acid, 2-Amino-3-methylbenzoic acid, 2-Amino-5-methylbenzoic acid, 2-Amino-6-methylbenzoic acid, 3-(Aminomethyl)benzoic acid hydrochloride, 3-Amino-2-methylbenzoic acid, 3-Amino-4-methylbenzoic acid, 4-(Aminomethyl)benzoic acid, 4-Amino-2-methylbenzoic acid, 4-Amino-3-methylbenzoic acid, 5-Amino-2-methylbenzoic acid, 3-Amino-2-naphthoic acid, 6-Amino-2-naphthoic acid, 2-Amino-3-nitrobenzoic acid, 2-Amino-5-nitrobenzoic acid, 4-Amino-3-nitrobenzoic acid, 5-Amino-2-nitrobenzoic acid, 3-(4-Aminophenyl)propionic acid, 3-Aminophthalic acid, 4-Aminophthalic acid, 3-Aminosalicylic acid, 4-Aminosalicylic acid, 5-Aminosalicylic acid, 2-Aminoterephthalic acid, 2-Amino-3,4,5,6-tetrafluorobenzoic acid, 4-Amino-2,3,5,6-tetrafluorobenzoic acid, (R)-2-Amino-1,2,3,4-tetrahydronaphthalene-2-carboxylic acid, (S)-2-Amino-1,2,3,4-tetrahydro-2-naphthalenecarboxylic acid, 2-Amino-3-(trifluoromethyl)benzoic acid, 3-Amino-5-(trifluoromethyl)benzoic acid, 5-Amino-2,4,6-triiodoisophthalic acid 2-Amino-3,4,5-trimethoxybenzoic acid, 2-Anilinophenylacetic acid, benzoyl phenylalanine (benzoyl-Phe), cyano phenylalanine (CN-Phe), Boc-2-Abz-OH, Boc-3-Abz-OH, Boc-4-Abz-OH, 2-(Boc-aminomethyl)benzoic acid, 3-(Boc-aminomethyl)benzoic acid, 4-(Boc-aminomethyl)benzoic acid, tert-Butyl 2-aminobenzoate, tert-Butyl 3-aminobenzoate, tert-Butyl 4-aminobenzoate, 4-(Butylamino)benzoic acid, 2,3-Diaminobenzoic acid, 3,4-Diaminobenzoic acid, 3,5-Diaminobenzoic acid, 3,5-Diaminobenzoic acid dihydrochloride, 3,5-Dibromoanthranilic acid, 3,5-Dichloroanthranilic acid, 4-(Diethylamino)benzoic acid, 4,5-Difluoroanthranilic acid, 4-(Dimethylamino)benzoic acid, 3,5-Dimethylanthranilic acid, 5-Fluoro-2-methoxybenzoic acid, Fmoc-2-Abz-OH, Fmoc-3-Abz-OH, Fmoc-4-Abz-OH, 3-(Fmoc-aminomethyl)benzoic acid, 4-(Fmoc-aminomethyl)benzoic acid, 4-(2-Fmoc-hydrazino)benzoic acid, 3-Hydroxyanthranilic acid, Methyl 3-aminobenzoate, 3-(Methylamino)benzoic acid, 4-(Methylamino)benzoic acid, Methyl 2-amino-4-chlorobenzoate, Methyl 2-amino-4,5-dimethoxybenzoate, 4-Nitroanthranilic acid, N-Phenylanthranilic acid and Sodium 4-aminosalicylate dehydrate.

In more particular embodiments of the first aspect, the Raman active tags are selected from the list consisting of the aromatic natural amino acids tyrosine, tryptophan, phenylalanine and non-natural amino acids benzoyl phenylalanine and cyano phenylalanine.

In yet another embodiment, a peptide is provided, wherein the peptide comprises a protease recognition sequence and at least 2 Raman active tags, wherein the at least 2 Raman active tags comprise at least 1 Raman active tag N-terminally from the protease recognition sequence and at least 1 Raman active tag C-terminally from the protease recognition sequence and wherein the peptide comprises SEQ ID NO:1: CALNN or an amino acid sequence having 90% or 100% homology to SEQ ID NO:2: CALNNXGGGG, wherein X can be any natural or non-natural aromatic amino acid, more particularly X can be Y, W, F, benzoyl-Phe or CN-Phe. In a more particular embodiment, the peptide has a maximum length of 35 amino acids or a length between 14 and 30 amino acids. In most particular embodiments, the peptide comprises the sequence CALNNXGGGGVRGNX (SEQ ID NO:6) or CALNXGGGGNNESXH (SEQ ID NO:7), wherein X can be Y, W, F or any non-natural aromatic amino acid, more particularly benzoyl-Phe or CN-Phe.

As used herein, the terms “peptide”, “polypeptide”, “protein” are used interchangeably and refer to a polymeric form of amino acids of any length, which can include coded and non-coded amino acids, natural and non-natural amino acids, chemically or biochemically modified or derivatized amino acids, and polypeptides having modified peptide backbones.

“Unnatural” amino acids or “non-natural” amino acids as used herein refer to the non-coded or non-proteinogenic amino acids that are not naturally encoded or found in the genetic code of any organism. An “unnatural” amino acid is thus any non-proteinogenic amino acid that is not one of the well-known 22 natural or proteinogenic amino acids that are used by the translational machinery to assemble proteins (H, D, R, F, A, C, G, Q, E, K, L, M, N, S, Y, T, I, W, P, V, U and formylmethionine). Non-natural amino acids either occur naturally or are chemically synthesized in the laboratory.

A “protease” also called a “peptidase” or “proteinase” as used herein refers to any enzyme that performs proteolysis, e.g., protein catabolism by hydrolysis of peptide bonds. In particular embodiments, “protease” refers to a peptide cleaving enzyme that recognizes a specific sequence in the peptide. This specific sequence is referred to as the “protease recognition sequence,” which is used herein as synonym for “protease cleaving sequence”.

“N-terminally from the protease recognition sequence” as used herein is a synonym of “before” or “left” or “north” from the protease recognition sequence and given that the peptide of the application is a linear molecule “N-terminally” thus also refers to the side of the protease recognition sequence where the peptide is attached to the surface or the carrier of the application that is described below.

As used herein, the terms “identical”, “similarity” or percent “identity” or percent “similarity” or percent “homology” in the context of two or more polypeptide sequences, refer to two or more sequences or subsequences that are the same or have a specified percentage of amino acid residues that are the same (e.g., 75% identity over a specified region) when compared and aligned for maximum correspondence over a comparison window or designated region as measured using sequence comparison algorithms or by manual alignment and visual inspection. Preferably, the identity exists over a region that is at least about 10 amino acids or nucleotides in length, or more preferably over a region that is 25-100 amino acids or nucleotides or even more in length.

The term “sequence identity” or “sequence homology” as used herein refers to the extent that sequences are identical on an amino acid by amino acid basis over a window of comparison. Thus, a “percentage of sequence homology” is calculated by comparing two optimally aligned sequences over the window of comparison, determining the number of positions at which the identical amino acid occurs in both sequences to yield the number of matched positions, dividing the number of matched positions by the total number of positions in the window of comparison (i.e., the window size), and multiplying the result by 100 to yield the percentage of sequence identity. A gap, i.e., a position in an alignment where a residue is present in one sequence but not in the other is regarded as a position with non-identical residues. Determining the percentage of sequence homology can be done manually, or by making use of computer programs that are available in the art. Examples of useful algorithms are PILEUP (Higgins & Sharp, CABIOS 5:151 (1989), BLAST and BLAST 2.0 (Altschul et al. J. Mol. Biol. 215: 403 (1990). Software for performing BLAST analyzes is publicly available through the National Center for Biotechnology Information.

In a second aspect, a carrier is provided having a molecule attached to it, the molecule comprises an enzyme recognition sequence and at least 2 Raman active tags, wherein the at least 2 Raman active tags comprise at least 1 Raman active tag before the enzyme recognition sequence and at least 1 Raman active tag behind the enzyme recognition sequence.

In one embodiment, a carrier is provided having a nucleic acid molecule attached to it, the nucleic acid molecule comprises a nuclease recognition sequence and at least 2 Raman active tags, wherein the at least 2 Raman active tags comprise at least 1 Raman active tag before the nuclease recognition sequence and at least 1 Raman active tag behind the nuclease recognition sequence.

As used herein, the term “nucleic acid”, “nucleic acid sequence”, “nucleic acid molecule” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The polynucleotide molecule may be linear or circular.

A “nuclease” as used herein refers to an enzyme capable of cleaving the phosphodiester bonds between monomers of nucleic acids. Nucleases variously effect single and double stranded breaks in their target molecules and act after recognizing specific nuclease recognition sequences present in the nucleic acid molecule.

In another embodiment, a carrier is provided having a peptide attached to it, the peptide comprises a protease recognition sequence and at least 2 Raman active tags, wherein the at least 2 Raman active tags comprise at least 1 Raman active tag before the protease recognition sequence and at least 1 Raman active tag behind the protease recognition sequence. In particular embodiments, a carrier is provided having any of the peptides described in the first aspect or in its accompanying embodiments attached to it.

In particular embodiments, multiple molecules can be attached to the carrier. The application thus also provides a carrier according to the above embodiments, to which a plurality of peptides and/or a plurality of nucleic acid molecules is attached wherein each of the peptides or nucleic acid molecules comprises a unique enzyme recognition site or wherein a plurality of the peptides or nucleic acid molecules comprises a plurality of unique enzyme recognition sites.

This is equivalent as saying that a carrier is provided on which a monolayer of molecules is bound, wherein the molecules contain a specific enzyme recognition site. The monolayer of molecules can consist of identical molecules, i.e., molecules comprising the same enzyme recognition sequence. In this case the carrier is useful to detect or quantify the presence, amount or activity of one particular enzyme. However, the carriers of current application can also be particularly useful for the parallel or multiplex detection of several nucleases or several proteases. Therefore, the monolayer of molecules consist of 2 or more different molecules or of 2 or more different groups of molecules. Thus, a carrier is provided having n different molecules attached to it, wherein the n different molecules differ from each other by comprising a different enzyme recognition sequence. This is equivalent as saying that every different molecule comprises a different or an unique enzyme recognition sequence or that the n different molecules represent n different groups of molecules, wherein the groups differ from each other by comprising a different or unique enzyme recognition sequence and wherein the molecules within one group comprise all the same enzyme recognition sequence. the carrier is further characterized by the fact that every molecule comprises at least one Raman active tag before and at least one Raman active tag behind the enzyme recognition sequence and wherein the ratios of the Raman intensity of the at least one Raman tag behind the enzyme recognition sequence and the Raman intensity of the at least one Raman tag before the enzyme recognition sequence or vice versa is specific for every molecule comprising a different enzyme recognition sequence or for every group of molecules having the same enzyme recognition sequence, and wherein n is an integer between 2 and 100. “Ratio” as used herein refers to the relationship between two or more Raman intensities, i.e., the Raman intensity of the at least one Raman tag before the enzyme recognition sequence and the Raman intensity of the at least one Raman tag behind the enzyme recognition sequence. In order to identify which molecule is cleaved by the to be tested protease or nuclease, the ratio is specific and thus a measure or a proxy for every molecule. However, it is irrelevant whether the ratio is calculated as the Raman intensity of the at least one Raman tag behind the enzyme recognition sequence over the Raman intensity of the at least one Raman tag before the enzyme recognition sequence or as the Raman intensity of the at least one Raman tag before the enzyme recognition sequence over the Raman intensity of the at least one Raman tag behind the enzyme recognition sequence as long as the numerator and denominator are consistently used as measure for one specific molecule. Therefore, the term “vice versa” (which means “or the other way around”) is incorporated in the embodiments.

In one embodiment, a carrier is provided having n different groups of molecules attached to it, wherein the n different groups differ from each other by comprising an unique enzyme recognition sequence, wherein the molecules within one group comprise the same enzyme recognition sequence, wherein every molecule comprises at least one Raman active tag before and at least one Raman active tag behind the enzyme recognition sequence, wherein the ratios of the Raman intensity of the at least one Raman tag behind the enzyme recognition sequence and the Raman intensity of the at least one Raman tag before the enzyme recognition sequence or vice versa is specific for every group of molecules comprising the same enzyme recognition sequence, and wherein n is an integer between 2 and 100. In a particular embodiment, n is an integer between 5 and 50 or between 4 and 30 or between 3 and 15 or between 2 and 10. Optionally, the carrier has also one or more spacer molecules attached to it. This spacer molecule does not comprise a Raman active tag but separates the Raman active tag comprising molecules attached to the carrier of the application from each other thereby improving the accessibility of the enzymes recognition sequence of the Raman active tag comprising molecules or increases the sensitivity by reducing the amount of substrate. In particular embodiments, the spacer molecule is a peptide with a length that is shorter than the peptide comprising the Raman active tag. In even more particular embodiments, the spacer molecule has a length of maximum 15 amino acids, maximum 10 amino acids or maximum 5 amino acids, even more particularly the spacer molecule comprises or consist of SEQ ID NO:1.

“Before” and “behind” the enzyme recognition sequence as used herein is equivalent to “left” and “right” or “north” and “south” or “preceding” and “following” the enzyme recognition sequence. In case of an amino acid sequence “before” and “behind” is also equivalent to “N-terminal” and “C-terminal”. In case of a nucleic acid sequence, “before” and “behind” is also equivalent to “5′ end” and “3′ end”.

As mentioned above, the n different molecules can also be seen as n different groups of molecules, wherein the groups differ from each other at least by having different enzyme recognition sequences but wherein the molecules within one specific group comprise that same enzyme recognition sequence. Therefore, in one embodiment, a carrier is provided having n different groups of molecules attached to it, wherein the n different groups differ from each other by comprising different enzyme recognition sequences, wherein the molecules within one group of molecules comprise the same enzyme recognition sequence, wherein every molecule of every group comprises at least one Raman active tag before and at least one Raman active tag behind the enzyme recognition sequence, wherein the ratios of the Raman intensity of the at least one Raman tag behind the enzyme recognition sequence and the Raman intensity of the at least one Raman tag before the enzyme recognition sequence or vice versa is specific for every group of molecules comprising a specific enzyme recognition sequence, and wherein n is an integer between 2 and 100. In a particular embodiment, n is an integer between 5 and 50 or between 4 and 30 or between 3 and 15 or between 2 and 10.

In a particular embodiment, the n different molecules are n different nucleic acid sequences and the enzymes are nucleases. In other particular embodiment, the n different molecules are n different amino acid sequences and the enzymes are proteases. In a more particular embodiment, the n different molecules comprise nucleic acid sequences and amino acid sequences and the carrier is provided to detect or quantify the presence, amount or activity of nucleases and proteases in parallel.

In another embodiment of the second aspect, the Raman active tag is a tag for surface enhanced Raman scattering (SERS) detection. In yet another embodiment, the Raman active tags are natural and/or non-natural aromatic amino acids. In a more particular embodiment, the Raman active tags are selected from the list consisting of the natural aromatic amino acids tyrosine, tryptophan and phenylalanine and the non-natural aromatic amino acids benzoyl-Phe and CN-Phe.

In yet another particular embodiment, a carrier is provided having a peptide attached to it, the peptide comprises a protease recognition sequence and at least 2 Raman active tags, wherein the at least 2 Raman active tags comprise at least 1 Raman active tag before the protease recognition sequence and at least 1 Raman active tag behind the protease recognition sequence, and wherein the peptide comprises SEQ ID NO:1: CALNN or an amino acid sequence having at least 90% or 100% homology to SEQ ID NO:2: CALNNXGGGG, wherein X can be any natural or non-natural aromatic amino acid, more particularly X can be Y, W, F, benzoyl-Phe or CN-Phe.

In yet another particular embodiment, a carrier is provided having n different groups of peptides attached to it, wherein the n different groups differ from each other by comprising different protease recognition sequences, wherein the peptides comprise SEQ ID NO:1: CALNN or an amino acid sequence having at least 90% or 100% homology to SEQ ID NO:2: CALNNXGGGG, wherein X can be any natural or non-natural aromatic amino acid, wherein the peptides within one group of peptides comprise the same protease recognition sequence, wherein every molecule of every group comprises at least one Raman active tag before and at least one Raman active tag behind the protease recognition sequence, wherein the ratios of the Raman intensity of the at least one Raman tag behind the protease recognition sequence and the Raman intensity of the at least one Raman tag before the protease recognition sequence or vice versa is specific for every group of peptides comprising the same protease recognition sequence, and wherein n is an integer between 2 and 100.

“Carrier” as used herein refers to a surface or a layer. The surface or layer is suitable to use in SERS detection. The layer can also be a multilayer, i.e., a layer that comprises several layers. In case of a multilayer, at least one layer should allow suitable SERS-detection. Therefore, according to particular embodiments, the carrier comprises a material selected from the list consisting of gold, silver, silicon, silicon-nitride, silicon dioxide, quartz, polystyrene, silica and dextran. In particular embodiments, the carrier is a nanoparticle, a nanodisk, a nanostructure, a chip.

In particular embodiments, the carrier is a planar substrate or a chip on which an optical waveguide is integrated or implemented. In particular embodiments, the optical waveguide is 2 or more optical waveguides. In more particular embodiments, the waveguides form a monolithic block on the carrier or chip. In even more particular embodiments, the integrated optical waveguide comprises a dielectric, transparent material with a high refractive index (here called n, n>1.6) on top of a silicon dioxide substrate on a silicon carrier. In even more particular embodiments, any of the carriers of the application is provided, wherein the carrier is a planar substrate on which a dielectric optical waveguide is integrated, wherein nanostructures are patterned on top of the integrated dielectric optical waveguide. In particular embodiments, the nanostructures are typically made of gold, or silver, copper or aluminum. The enhanced electromagnetic field close to these structures gives rise to surface-enhanced Raman scattering. In yet another more particular embodiments, the integrated optical waveguide comprises a thin layer (thickness between 100 and 350 nm) of silicon nitride guiding the electromagnetic wave, patterned on top of a silicon dioxide substrate on a silicon carrier.

In a third aspect, the use of any of the carriers according to the second aspect or one of its embodiments is provided to detect or quantify the presence, amount or activity of at least one enzyme in a biological sample. Also the use of any of the peptides according to the first aspect or one of its embodiments is provided to detect or quantify the presence, amount or activity of at least one enzyme in a biological sample. As mentioned in the third aspect, carriers of current application are particularly useful for the detection or quantification of the activity of multiple enzymes in parallel or simultaneously in one biological sample. Detection of multiple molecules simultaneously is also known as multiplexing. Therefore, the use of any of the carriers according to the second aspect or one of its embodiments is provided to simultaneously detect or quantify the presence, amount or activity of at least two enzymes in one biological sample. To detect at least two enzymes in parallel, the carrier comprises at least two molecules comprising different enzyme recognition sequences, wherein the sequences are recognized by the enzymes that need to be detected or quantified or the carrier comprises at least two groups of molecules wherein the groups differ from each other by comprising different enzyme recognition sequences, wherein the sequences are recognized by the enzymes that need to be detected or quantified, wherein the molecules within one group comprise the same enzyme recognition sequence.

Several diseases and disorders can be diagnosed by the enhanced presence or enhanced activity of one or more proteases. A non-limiting example is the detection of Prostate Specific Antigen (PSA) for diagnosing prostate cancer (U.S. Pat. No. 8,685,743 B2, incorporated by reference herein). In addition to detection of enhanced protease activity, reduced protease activity is also clinically relevant. The carriers of the present application are particularly useful to detect or quantify the presence, amount or activity of proteases. Therefore, the carriers of the second aspect and of any of its embodiments, are provided for use as diagnostic.

A “biological sample” as used herein refers to a sample or specimen taken from a subject. In particular embodiments, the subject is a mammal, more particularly a human. In particular embodiments, a biological sample is selected from a blood sample, a serum sample, a urine sample, a stool sample, an oral sample, a mucosal biopsy sample, a sample of the lumen content.

In a fourth aspect, a method is provided for detecting or quantifying the presence, amount or activity of an enzyme in a biological sample, the method comprising:
a. contacting one of the carriers according to the second aspect or one of its embodiments with the sample, wherein the molecule attached to the carrier comprises a recognition sequence for the enzyme;
b. determining the Raman ratio before and after contacting the sample with the carrier, wherein the Raman ratio is the ratio between the Raman intensity of the one or more Raman active tags behind the enzyme recognition sequence of the molecule and the Raman intensity of the one or more Raman active tags before the enzyme recognition sequence of the molecule or vice versa;

wherein the change in Raman ratio before and after contacting the sample with the carrier gives a metric for the presence, amount or activity of the enzyme in the sample.

In case the molecule comprising the enzyme recognition sequence (and thus attached to the carrier from this document) comprises more than one Raman active tag before or after the enzyme recognition sequence, then the Raman ratio is determined using the sum of the Raman intensities of the more than one Raman active tag before or after the enzyme recognition sequence.

“Recognition sequence” or “enzyme recognition sequence” is used herein as equivalent to “cleaving sequence”.

Also provided are solutions for the multiplex detection or multiplex quantification of the presence, amount or activity of multiple enzymes. More particularly, a method is provided for the multiplex detection or multiplex quantification of the presence, amount or activity of n different enzymes in one biological sample, the method comprising:
a. Contacting the carrier according to the second aspect or one of its embodiments with the sample, wherein the n different molecules attached to the carrier comprise the recognition sequences for the n different enzymes;
b. Determining the Raman ratio before and after contacting the sample with the carrier, wherein the Raman ratio is the ratio between the Raman intensity of the one or more Raman active tags behind the enzyme recognition sequence of the n different molecules and the Raman intensity of the one or more Raman active tags before the enzyme recognition sequence of the n different molecules or vice versa;

wherein for each different enzyme and for the Raman active tags of a molecule comprising the recognition sequence of the enzyme, the difference in Raman ratio before and after contacting the sample with the carrier gives a metric for the presence, amount or activity of the n different enzyme in the sample.

In particular embodiments, the enzyme is a protease and the recognition sequence is a protease recognition sequence or the enzyme is a nuclease and the recognition sequence is a nuclease recognition sequence. The change in Raman ratio will be a read-out for protease and/or nuclease activity within the sample. In particular embodiments, the change is an at least 10%, an at least 20%, an at least 30%, an at least 40% or an at least 50% reduction or increase in Raman ratio after contacting the carriers of the application with a biological sample compared to the Raman ratio before contacting the carriers of the application with a biological sample.

In Example 8, it is demonstrated that Raman spectra can be detected when Raman scatterers are excited with waveguides. Therefore, according to another embodiment, the above mentioned methods are provided, wherein the Raman scattering is excited and/or collected by one or more waveguides. In particular embodiments, the waveguides are optical waveguides integrated on the carrier.

An “optical waveguide” as used herein refers to a structure guiding a confined electromagnetic wave with a visible or near-infrared wavelength (500-1600 nm).

An “integrated optical waveguide” as used herein refers to the implementation of an optical waveguide on a planar substrate, here referred to as chip. The waveguides form a monolithic block on the chip.

In a typical configuration, the integrated optical waveguide comprises a dielectric, transparent material with a high refractive index (here called n, n>1.6) on top of a silicon dioxide substrate on a silicon carrier. These are referred to as integrated dielectric optical waveguides. In particular embodiments, the application thus also provides the methods of the application wherein the Raman scattering is excited and/or collected by one or more integrated optical waveguides, wherein the integrated optical waveguides are integrated dielectric optical waveguides.

In a specific configuration, SERS-nanostructures are patterned on top of this integrated dielectric optical waveguide. These structures are typically made of gold, or silver, copper or aluminum. The enhanced electromagnetic field close to these structures gives rise to surface-enhanced Raman scattering. Thus in a more particular embodiment, the Raman scattering is excited and/or collected by one or more integrated dielectric optical waveguides, wherein the waveguides are patterned with gold, silver, copper or aluminum nanostructures. In another particular embodiment, the integrated optical waveguide comprises a thin layer (thickness between 100 and 350 nm) of silicon nitride guiding the electromagnetic wave, patterned on top of a silicon dioxide substrate on a silicon carrier.

The means and methods disclosed in current application can be used in diagnostics but also for drug discovery. The application thus also provides a method to screen for compounds with protease or nuclease activity, the method comprising:
a. contacting one of the carriers of the second aspect or of one of its embodiments with at least one test compound;
b. measuring the Raman intensities of the at least two Raman active tags of the one or more different molecules attached to the carrier before and after contacting the test compound with the carrier;
c. identifying the test compound as a compound with protease or nuclease activity, if at least one Raman ratio decreases with at least 25% after contacting the carrier with the test compound, wherein the Raman ratio is the ratio between the Raman intensity of the one or more Raman active tags behind an enzyme recognition sequence of a molecule attached to the carrier and the Raman intensity of the one or more Raman active tags before the enzyme recognition sequence or vice versa.

In particular embodiments, the at least one Raman ratio should decrease with at least 30%, at least 40%, at least 50%, at least 55%, at least 60%, at least 65%, at least 70%, at least 75%, at least 80%, at least 85%, at least 90%, at least 94% or 100% to identify the test compound as a compound with protease or nuclease activity. In other particular embodiments, the at least one Raman ratio should decrease at least 2-fold, at least 3-fold, at least 4-fold, at least 5-fold, or at least 10-fold.

The application also provides a method to screen for compounds that inhibit the activity of a protease or nuclease, the method comprising:
a. contacting one of the carriers of the second aspect or of one of its embodiments with at least one test compound;
b. measuring the Raman intensities of the at least two Raman active tags of the one or more molecules attached to the carrier before and after contacting the test compound with the carrier in the presence of the protease or nuclease, wherein the one or more molecules comprises a recognition sequence for the protease or nuclease;
c. identifying the test compound as a compound that inhibits activity of the protease or nuclease, if the Raman ratio in the presence of the test compound is at least 25%, at least 35%, at least 45%, at least 55%, at least 65%, at least 75%, at least 85%, at least 100% higher than the Raman ratio in the absence of the test compound, or identifying the test compound as a compound that inhibits activity of the protease or nuclease, if the Raman ratio in the absence of the test compound is at least 25%, at least 35%, at least 45%, at least 55%, at least 65%, at least 75%, at least 85%, at least 100% lower that the Raman ratio in the presence of the test compound, wherein the Raman ratios are the ratios between the Raman intensity of the one or more Raman active tags behind an enzyme recognition sequence of a molecule attached to the carrier and the Raman intensity of the one or more Raman active tags before the enzyme recognition sequence.

The following examples are intended to promote a further understanding of the present disclosure. While the present disclosure is described herein with reference to illustrated embodiments, it should be understood that the disclosure is not limited hereto. Those having ordinary skill in the art and access to the teachings herein will recognize additional modifications and embodiments within the scope thereof. Therefore, the present disclosure is limited only by the claims attached herein.

### EXAMPLES

### Example 1: Measurement Principle and Design of the Peptide Substrate

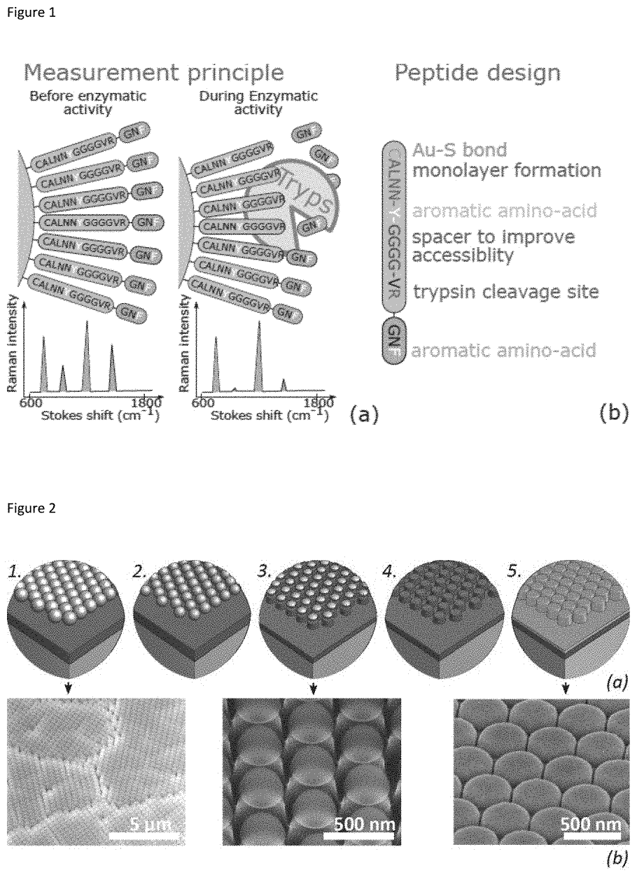

FIG. 1(a) illustrates the disclosure. A monolayer of peptides containing a specific cleavage site was bound to a gold nanostructure. This monolayer provides a SERS signature from amino acids both before and behind the cleavage site. After protease digestion, the part of the SERS spectrum originating from the cleaved-off, non-surface bound product decreases, while the products attached to the gold surface provide a steady signal. Although the measurement principle is fairly straightforward, a correct design of the peptide is crucial for efficient protease digestion and sensitive SERS detection. The peptides should form a stable monolayer on a gold surface, provide an accessible cleavage site and include strong SERS scatterers. Furthermore, the total length of the molecule must remain limited to a few nanometers because of decreasing SERS signal with increasing distance (refs 24, 25). FIG. 1(b) shows the proposed peptide in more detail, designed as a specific substrate for trypsin digestion. From amine- to carboxyl-terminus, it starts with the pentapeptide CALNN (SEQ ID NO:1) (refs 20, 22, 23, 26). In this part, the cysteine (C) ensures a covalent sulphur-gold bond (ref 27). The hydrophobic amino acids alanine (A) and leucine (L) help to form a self-assembled monolayer, followed by a double hydrophilic asparagine (N) to ensure a good solubility (ref 20). CALNN (SEQ ID NO:1) is followed by tyrosine (Y), an aromatic amino acid that serves as a first SERS reporter. If present, its SERS signature confirms that the CALNNY (SEQ ID NO:) fraction is still bound to the gold surface, meaning that there has not been any ligand exchange (refs 28), desorption or non-specific cleavage in this part. Next, a stretch of glycines (GGGG (SEQ ID NO:9)) is included, small amino acids that form a flexible chain to improve the accessibility for the protease to the cleavage site. It was experimentally found that the presence of this additional spacer is crucial for trypsin activity on surface-bound peptides. These glycines are followed by VR being, respectively, the P2 and P1 subsite for trypsin. It is well known that trypsin cleaves at the carboxyl side of arginine (R) and lysine (K) (ref 29). The hydrophobic valine (V) in a P2 position further increases the efficiency of the catalysis (ref 30). The cleavage site is followed by a GNF sequence. The small glycine at P1′ ensures a good accessibility of trypsin to the cleavage site. The hydrophilic asparagine helps the solubility of the end-fraction of the peptide and, finally, the second aromatic amino acid phenylalanine (F) functions as a second SERS reporter. As a consequence, for this specific substrate the intensity of F-related peaks divided by that of Y-related peaks in the SERS spectrum (I_{F}/I_{Y}) gives a metric for cleavage and diffusion of the -GNF fraction.

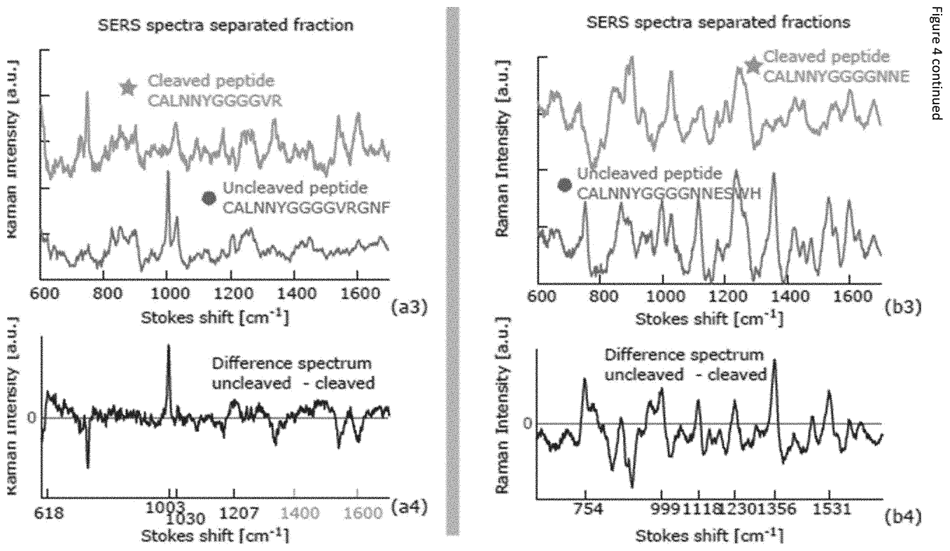

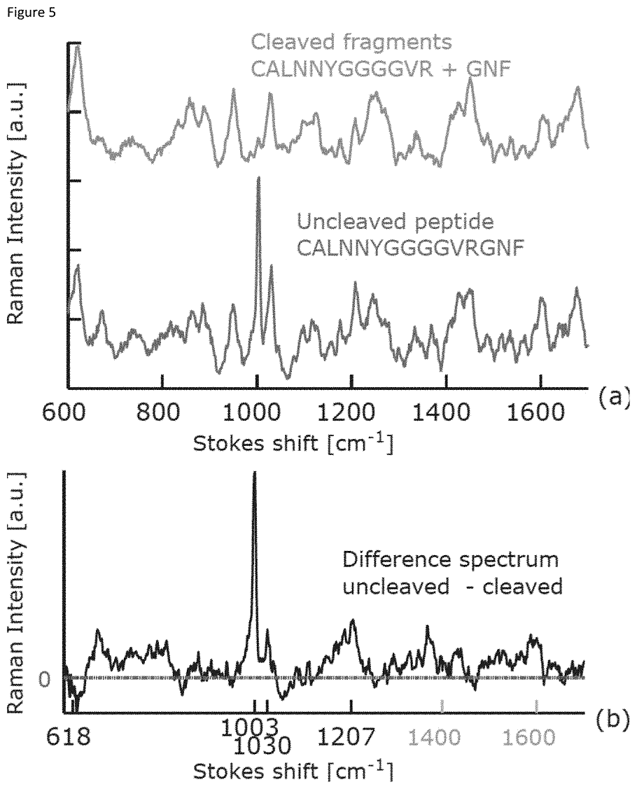

An analogous peptide substrate for a different protease was fabricated by adjusting the specific cleavage site. Placing the amino acids NNE- in the P3-P1 and -SWH in P1′-P3′ positions (FIG. 4(b1)) makes the substrate suited for hydrolysis by endoproteinase Glu-C, which cleaves peptide bonds C-terminal to glutamic acid (E) residues (ref 31). Tryptophan (W) serves here as an aromatic reporter in the cleaved-off fraction, resulting in a different SERS spectrum that allows using I_{W}/I_{Y} as a metric for endoproteinase Glu-C digestion. Thus, the generic design of the peptide substrate provides the ability to use this technology in different applications, and has the potential for simultaneously monitoring the activity of different proteases on different substrates.

### Example 2: The Nanodome SERS Platform as a Compromise Between Accessibility and Enhancement

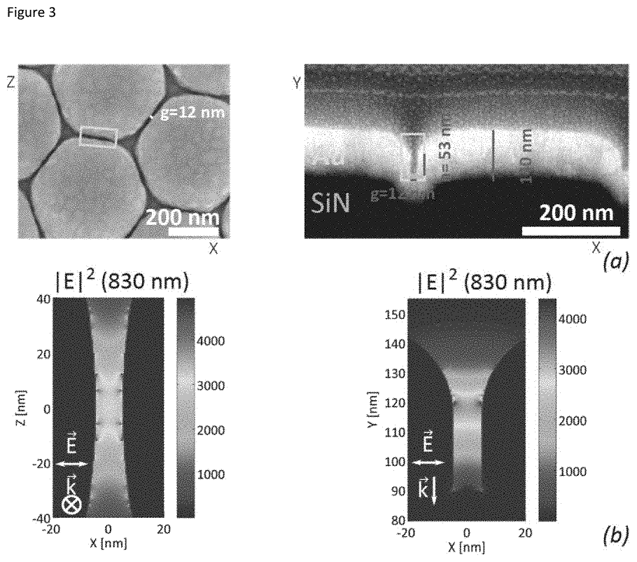

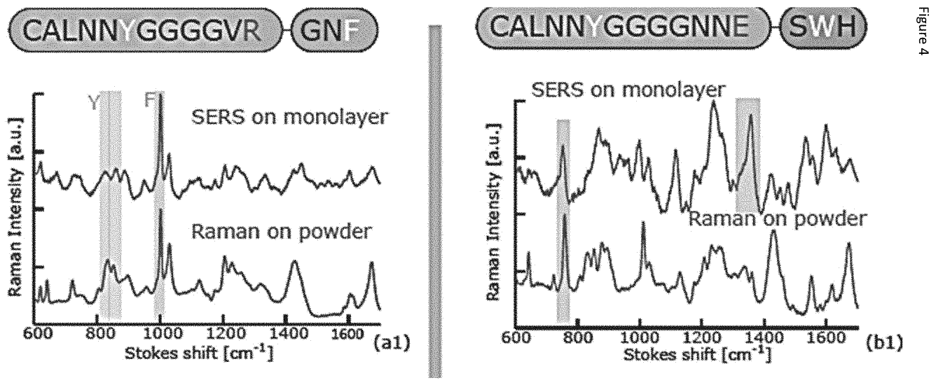

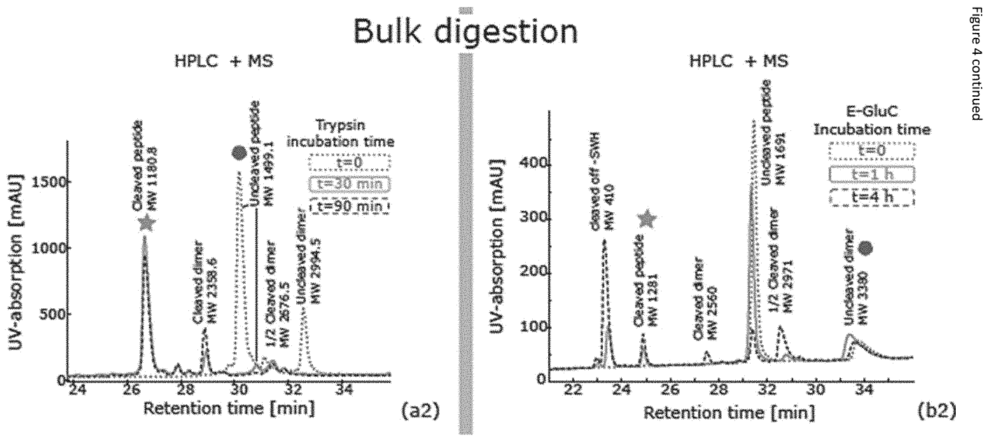

For most applications, the ideal SERS platform provides a strong, uniform field-enhancement and has good batch to batch reproducibility. For the specific case of a sensitive and quantitative monitoring of protease activity, the field enhancement has to stretch a few nanometers from the gold surface and the hotspots have to be accessible. The latter is of crucial importance, as most proteases have a molecular weight of 20-50 kDa, roughly corresponding to a Stokes radius of 2-3 nm (ref 32). A peptide substrate located in a <5 nm wide hotspot is inaccessible for a trypsin (23 kDa) or endoproteinase Glu-C (27.7 kDa) molecule. Fortunately, pore sizes starting from 10 nm are accessible to enzymes in this size range (ref 33). The requirements described above are to a certain degree contradictive. Isolated nanostructures such as nanorods or nanotriangles provide optimal accessibility, but exhibit a low enhancement factor and a stronger distance-dependence as compared to coupled geometries. On the other hand, superior surface-enhancement is achieved in coupled nanostructures with a sub-5 nm, poorly accessible gap (ref 34). To address these concerns, gold-nanodome structures (ref 21) were fabricated with a gap (g) of 10-15 nm. FIG. 2 shows the fabrication flow and corresponding SEM images of these nanodome patterned chips, processed on 4″ wafer using nanosphere-lithography. This fabrication method provides ample chips at a reasonable cost and effort. It avoids using e-beam lithography (ref 11) while offering a superior control on the hotspot size and enhancement factor as compared to colloidal approaches (ref 35). The localized surface-plasmon resonance of these nanodome structures was optimized through UV-Vis reflection and SERS measurements for an optimal enhancement with a 785 nm Raman pump laser and 600 cm⁻¹ to 1700 cm⁻¹ Stokes shifts in a water environment. FIG. 3(a) shows the resulting geometry with a gap width (g) of 12±2 nm and a height (h) of 53±4 nm. A SERS substrate enhancement factor (SSEF ref 36, see experimental section) of 1±0.2×10⁶ was experimentally measured for the chips used in this application. The coefficient of variation (σ/μ) on the SERS signal across a single chip is 6-7%. For comparison, a measurement was taken of a maximum SSEF of 6×10⁶ in nanodome structures with a gap width of approximately 6±2 nm, unlikely to have a good accessibility of the peptide substrate in the hotspots. Note that SSEF is a figure for the surface-enhancement per molecule averaged across the gold surface. The surface area of the hotspots accounts for approximately 10-15% of the total gold surface area, calculated from the SEM images in FIG. 3(a). Thus, for molecules in this region, the SERS hotspot enhancement factor (SHEF) is in the order of 10⁷, which is in correspondence with the 3D FDTD simulated electric field profile (FIG. 3b) in a |E|⁴ approximation. These field profiles also show that the LSPR stretches across the full width of the gap. Assuming a loading density of 2 peptides/nm (ref 2, 26, 28) there are roughly 10⁴ peptides per hotspot.

### Example 3: Hydrolysis of Unbound Peptides Observed Through SERS Spectra of HPLC Separated Fractions

FIG. 4(a1,b1) compares the spontaneous Raman- to SERS-spectra of CALNNYGGGGVRGNF (SEQ ID NO:3) (trypsin substrate) and CALNNYGGGGNNESWH (SEQ ID NO:4) (endoproteinase Glu-C substrate) peptides labelled on a nanodome platform. Their respective Raman bands are identified in Table 1 and Table 2 based on SERS (refs 18, 19) and Raman (refs 37, 38) spectra of amino acids reported in literature and on own measurements of the peptide CALNN. It is of particular interest the peaks selective for the peptide products that are either cleaved off or remain on the surface. These include the 1003 cm⁻¹ symmetric bending mode of phenylalanine (F) versus the 833 cm⁻¹ in plane and 853 cm⁻¹ out plane ring breathing modes of tyrosine (Y), and the symmetric benzene/pyrrole in-phase breathing mode of tryptophan (W) at 760 cm⁻¹ (ref 18).

As a first step, the activity of the protease on the substrate in solution was monitored. RP-HPLC was used to separate the substrate from the digestion products, which are subsequently labelled on a nanodome platform. From the difference spectrum, the characteristic peaks of the cleaved-off and remaining fractions are derived. FIG. 4(a2-a4) depicts the results of this experiment for trypsin and its CALNNYGGGGVRGNF (100 μg/ml) (SEQ ID NO:3) substrate. The peptide was incubated with trypsin (3.3 μg/ml) at a 1/30 (w/w) ratio in a 50 mM ammonium-bicarbonate (pH 7.8) buffer in water, and separated with RP-HPLC (FIG. 4(a2)) after 0, 30 and 90 minutes of incubation. The RP-HPLC-separated peptides and their fragments were identified using MALDI-TOF mass spectrometry. Prior to adding trypsin, it was found that two fractions; one corresponding to an uncleaved monomer and the other to an uncleaved dimer. The dimer formation is a consequence of the oxidation of the cysteine thiol groups upon which a disulphide bond is formed between two peptides. The substrate was almost fully digested after 30 minutes and transformed into cleaved monomer, single cleaved dimer and double cleaved dimer fractions. After 90 minutes, the peptides were found to be fully cleaved. Subsequently, nanodome chips were labeled with cleaved and uncleaved fractions. The resulting SERS spectra (FIG. 4(a3 -a4)) show a full disappearance of the 1003 cm⁻¹ peak in the cleaved fraction. Furthermore, the peaks at 618 cm⁻¹, 1030 cm⁻¹ and 1207 cm⁻¹ show a partial decrease, although their signal to noise ratio is low. All these peaks correspond to those attributed to phenylalanine (Table 1). From this experiment, it is concluded that the ratio between the 1003 cm⁻¹ peak and the 829-860 cm⁻¹ peaks (I₁₀₀₃/I₈₂₉₋₈₆₀) is a correct metric for I_{F}/I_{Y}, the cleavage of the substrate by trypsin.

TABLE 1
Peak assignment of Raman and SERS spectra of the CALNNYGGGGVRGNF
(SEQ ID NO: 3) peptide. The Raman spectrum of the short peptide
CALNN (SEQ ID NO: 1) was separately measured. SERS peaks marked in
bold will decrease upon trypsin activity. Peaks in italic
originate from peptide backbone vibrations

| Raman | SERS | | | | |
|---|---|---|---|---|---|
| (cm⁻¹) | (cm⁻¹) | Origin | Raman | SERS | Origin |
| 621 | 618 | F¹⁸ | 1126 | 1124 | G³⁷, CALNN |
| | | | | | (SEQ ID |
| | | | | | NO: 1) |
| 642 | — | Y¹⁸, CALNN | 1176 | 1178 | Y³⁷ |
| 723 | 730 | CALNN | 1207 | 1206 | F¹⁸ |
| 834 | 829 | Y¹⁸ | 1229 | 1240 | Amide III β⁴⁴ |
| 853 | 860 | Y¹⁸, A³⁷ | 1325 | 1330 | G³⁷ |
| 898 | 889 | G³⁷ | 1429 | 1426 | CALNN (SEQ |
| | | | | | ID NO: 1) |
| 957 | 948 | CALNN | — | 1448 | G³⁷ |
| 1003 | 1003 | F^{18,37} | 1607 | 1603 | CALNN (SEQ |
| | | | | | ID NO: 1) |
| 1031 | 1030 | F^{18,37}, CALNN | 1674 | 1677 | Amide I |

TABLE 2
Peak assignment for the CALNNYGGGGNNESWH (SEQ ID NO: 4) peptide
for endoproteinase Glu-C digestion. SERS peaks marked in bold will
decrease upon endoproteinase Glu-C activity.

| Raman | SERS | | | | |
|---|---|---|---|---|---|
| (cm⁻¹) | (cm⁻¹) | Origin | Raman | SERS | Origin |
| 642 | 642 | Y¹⁸, CALNN | - | 1118 | W³⁷ |
| 723 | — | CALNN | 1176 | 1177 | Y³⁷ |
| 758 | 754 | W^{18,37,38} | 1232 | 1235 | W^{37,38}, |
| | | | | | Amide |
| | | | | | IIIB⁴⁴ |
| 832 | — | Y¹⁸ | 1257 | — | W³⁷, H³⁷ |
| 852 | | Y¹⁸, A³⁷ | 1361 | 1356 | W^{18,37,38} |
| | | | | | CALNN |
| | | | | | (SEQ ID |
| | | | | | NO: 1), |
| — | 868 | ? | 1430 | 1423 | W³⁷ |
| 879 | — | W^{18,37,38} | — | 1535 | ? |
| 893 | — | G³⁷ | 1552 | — | W^{18,37,38} |
| — | 999 | | — | 1602 | CALNN |
| | | | | | (SEQ ID |
| | | | | | NO: 1) |
| 1012 | — | W^{18,37} | 1618 | — | W³⁷, |
| | | | | | G³⁷, |
| | | | | | CALNN |
| | | | | | (SEQ ID |
| | | | | | NO: 1) |
| 1030 | 1027 | CALNN (SEQ ID | 1672 | 1674 | Amide I |
| | | NO: 1) | | | |

FIG. 4(b2-b4) describes an analogous experiment for endoproteinase Glu-C (3.3 μg/ml) and its CALNNYGGGGNNESWH (100 μg/ml) substrate (SEQ ID NO:4). Peptide digestion was slightly less efficient, with almost full conversion of substrate to products only after 4 hours of incubation. Because of the large adsorption of tryptophan at 270 nm and its strong interaction with the RP-HPLC resin, the −SWH product is also visible as a separate fraction. After binding the cleaved peptide and uncleaved dimer to nanodome chips, a disappearance of the 754 cm⁻¹ and 1356 cm⁻¹ peaks upon cleavage was observed, as well as a reduction of the contributions at 999, 1118 and 1531 cm⁻¹ peaks. These changes correspond to the peak assignments shown in Table 2.

### Example 4: Hydrolysis of Unbound Peptides Observed Through SERS Spectra of a Non-Separated Mixture

Apart from a thiol-gold interaction, N-terminal primary amines can also lead to a charge-based adsorption on metal surfaces (ref 39). This effect may be additive in the case of cysteine, but could lead to the adsorption of unwanted amino acids on the gold surface. In a second set of in-solution experiments, a similar trypsin was run—CALNNYGGGGVRGNF (SEQ ID NO:3) assay without separating the fractions by RP-HPLC (FIG. 5). One nanodome chip was labelled with a reference solution of the peptide (100 μg/ml), another with a solution of peptide (100 μg/ml) and trypsin (2 μg/ml) after 2 hours of incubation. The lack of a phenylalanine peak at 1003 cm⁻¹ in the trypsin-incubated solution confirms that the peptide had been fully digested and that the products bind the surface through the thiol side-chain on the cysteine, but not through charge-based adsorption of the free amino group on the −GNF cleaved off product. Furthermore, the SERS and difference SERS-spectra shown in FIG. 5 confirm the findings of the RP-HPLC experiment in FIG. 4(a).

### Example 5: SERS-Monitored Trypsin Hydrolysis of Gold-Nanodome Bound Peptides

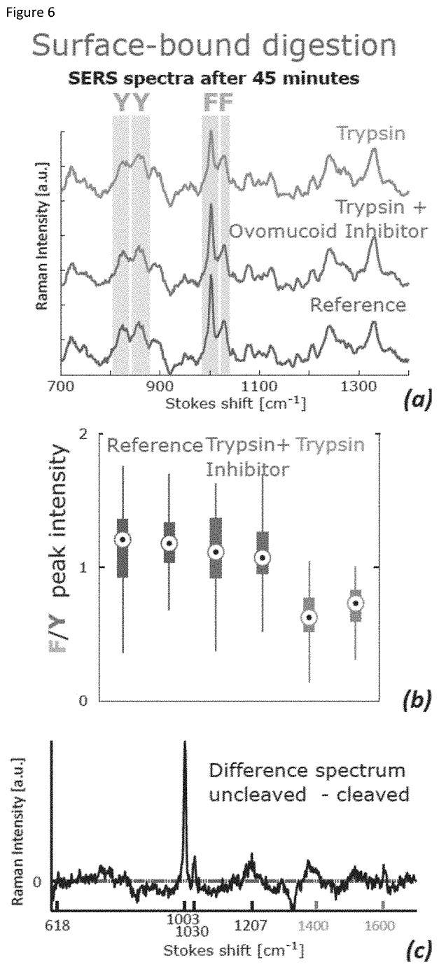

In a third experimental setup, three CALNNYGGGGVRGNF (SEQ ID NO:3) labelled nanodome chips were incubated in a buffer without trypsin, with trypsin and with trypsin plus an ovomucoid inhibitor for 45 minutes (FIG. 6). The chip incubated with trypsin plus inhibitor shows a SERS spectrum resembling that of the reference chip, suggesting full blockade of tryptic cleavage by the ovomucoid inhibitor. On the chip with trypsin only, the surface bound peptides are cleaved, resulting in a decrease of 11003/1829-860 by 40-48%. A full disappearance of the −GNF fingerprint was never observed, at most 11003/1829-860 decreased by 53% over all of the experiments. Two possible origins are considered for the remaining signal at 1003 cm⁻¹. One is a partial re-adsorption of the −GNF products on the gold surface; the tripeptides that do not diffuse out of the hotspot region will continue contributing to the SERS spectrum. However, the experiment in FIG. 5 suggests this is not the case. More likely, the remaining signal originates from peptides that are not accessible to the protease due to steric hindrance. This can be a result of a too dense monolayer of peptides (refs 11, 23) or inaccessible parts of the nanodome geometry. Especially in the latter case, the inaccessible peptides are probably located in the nanodome gaps and will contribute disproportionally strong to the SERS signal. Thus, it is plausible that significantly more than 53% of the protease-accessible peptides has been cleaved.

The difference spectrum in FIG. 6(c) shows exactly the same features as those observed upon bulk digestion followed by SERS labelling in FIG. 4(a4) and FIG. 5(c). Furthermore, significant differences in the absolute strength of the CALNNYGGGGVR (SEQ ID NO:5) fingerprint were not observed, which shows that ligand exchange was limited in this assay. Ligand exchange is more prominent in a reducing environment, in which a reduction of the gold-sulphur bond results in a detachment of the peptides. These reductive environments are present in cells and of particular importance for the activation of cysteine proteases. While the normalization to the trypsin peaks at 833-853 cm⁻¹ is not strictly necessary in this case, it does provide an inherent correction for differences in acquisition parameters such as laser power or focus drift as well as limited variations across the SERS platform.

### Example 6: Real Time Observation of Trypsin Activity

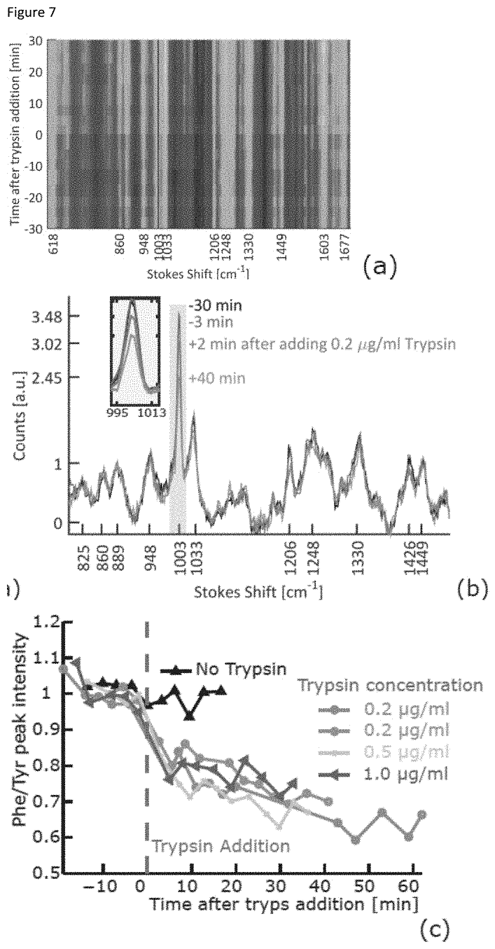

Finally, CALNNYGGGGVRGNF (SEQ ID NO:3) labelled chips were incubated with trypsin under the Raman microscope for a real-time acquisition of SERS spectra. FIG. 7(a) shows the evolution of these spectra before and after trypsin addition. A new spectrum was acquired every 2-3 minutes with an integration time of 100 seconds. A relative decrease of I₁₀₀₃ versus the peaks at 829, 860, 948, 1248, 1330, 1603 and 1677 cm⁻¹ is visible within the first minutes after adding 0.2 μg/ml trypsin (8.6 nM) to a total volume of 1 ml (8.6 pmol), in agreement with Table 1 and the earlier experiments described in this work. SERS spectra after 2 minutes and 30 minutes of trypsin incubation show a further reduction of I₁₀₀₃ and I₁₂₀₆ with increasing incubation time (FIG. 7 (b)). The fast decrease of I₁₀₀₃/I₈₂₉₋₈₆₀ in the first minutes after trypsin addition demonstrates that at trypsin concentrations ranging from 1 to 0.2 μg/ml, most of the accessible substrate was cleaved within the time-span of the first measurement (FIG. 7 (c)). The curves of 11003/1829-860 versus time qualitatively agrees to the kinetics of enzymatic reactions on self-assembled monolayers (refs 40, 41). Remarkably, the SERS spectra suggest that the cleavage rate is similar for 0.2, 0.5 and 1 μg/ml trypsin. It is assumed that even for the lowest trypsin concentration, a limiting velocity for cleavage of the surface-bound peptides in the plasmonic hotspots is reached. Enzyme kinetics on immobilized substrates can be substantially different from those on substrates in solution. The latter follow Michaelis-Menten kinetics under the assumption of an excess substrate concentration. On the nanodome surface this is no longer valid because of the very low concentration of immobilized substrates, therefore a further increase in enzyme concentration does not increase the initial cleavage rate (ref 41).

More measurements are required to support this assumption and accurately determine the trypsin detection limit and on-chip enzyme kinetics, especially in the first minutes after trypsin incubation.

### Example 7: Multiplexed Detected of Protease Activity

To simultaneously monitor the activity of multiple proteases on a single spot, the gold surface is functionalized with different peptides, each a specific substrate for one protease. These peptides differ on at least two locations: (1) the amino acids that constitute the protease-specific recognition site and (2) the reporter molecule that provides characteristic SERS peaks. Examples of different recognition sites include (from p4-p1 position) xxxR- for Trypsin, xxxE—for endoproteinase GluC or YVAD- (SEQ ID NO:13) for caspase-1. The reporter molecule can be a natural aromatic amino acid (Phe, Tyr or Tryp), a non-natural aromatic amino acid or another end-chain modification such as -AMC or -pNA. Important is that each of these molecules provide a clearly distinguishable SERS spectrum.

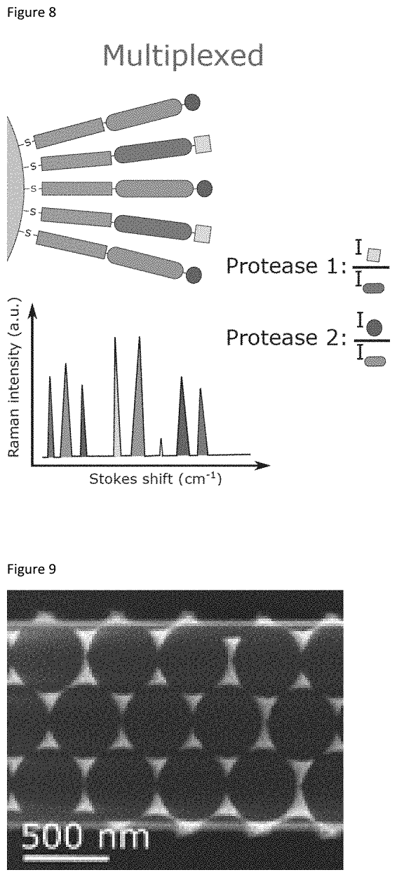

It is also possibly to vary the aromatic molecule generating a distinguishable SERS signal of the fraction remaining on the gold substrate after cleavage. The shorter the distance between both aromatic reporters, the higher the probability to distinguish specific protease activity from unspecific cleavage. If this reporter is kept the same (for example, a Phe in the remaining fraction of each peptide), it will still serve as a control against ligand exchange. The part of the peptide forming the self-assembled monolayer on the gold layer does not change (here -CALNN (SEQ ID NO: 1) or -CCALNN (SEQ ID NO: 10), or alternatively an alkanethiol-PEG chain). Prior to labelling the gold surface, the different peptides are mixed in appropriate ratios. Next, the gold surface is incubated with this mixture, which results in the formation of a heterogeneous monolayer of peptides (FIG. 8). The activity of a specific protease is monitored from the ratio of the SERS-bands originating from the cleaved of fraction of its specific substrate over the SERS-bands of the part that remains bound to the gold. This provides an independent metric for each different protease. Because of the spectrally narrow optical fingerprint, tens of different peptides (and thus proteases) can be monitored simultaneously.

### Example 8: Inclusion of Non-Natural Aromatic Amino Acids for Multiplexing Perspective

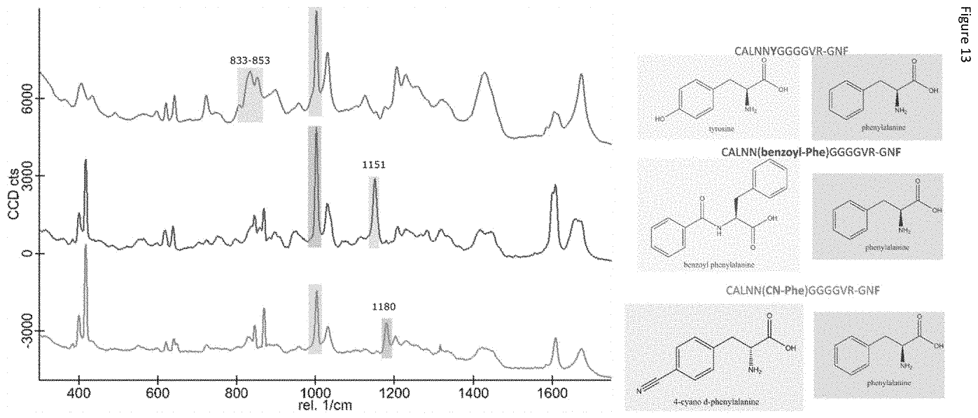

Because of its specificity, Raman spectroscopy has large potential for a dense spectral multiplexing of protease activity measurement. In the measurement described above, the specific SERS signal of the peptide is provided by natural aromatic amino acids. As there are only three of these (tyrosine, tryptophan and phenylalanine), an extension of this set is necessary to enable n>2 multiplexed measurements. For this purpose, the large variety of commercially available unnatural aromatic amino acids can be made use of. These can be incorporated in the peptide chain using conventional on-bead synthesis methods. To demonstrate this principle, two analogue peptides to the earlier described peptide (CALNNYGGGGVRGNF) (SEQ ID NO:3) were synthesized. The tyrosine was replaced by, respectively, 4-cyano-phenylalanine (CALNN(cnF)GGGGVRGNF) (SEQ ID NO:11) and benzoyl-phenylalanine (CALNN(bzF)GGGGVRGNF) (SEQ ID NO:12). FIG. 13 shows the Raman spectrum of these peptides in lyophilized state, with selected specific peaks for the different aromatic amino acids highlighted.

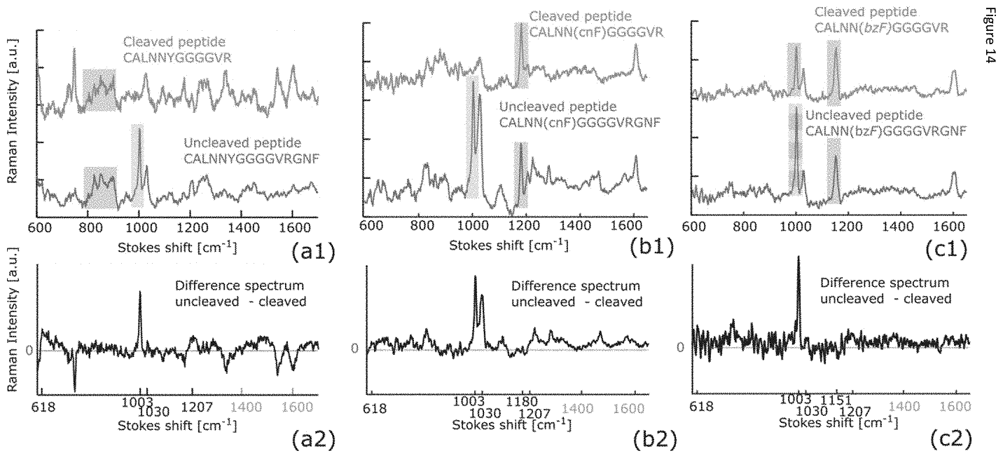

The activity of trypsin on the peptides with unnatural aromatic amino acids was verified using HPLC/mass spectrometry, showing complete digestion of 100 μg/ml peptide after 1 hour incubation with a 4 μg/ml Trypsin concentration. Subsequently, cleaved and uncleaved fractions were labelled on a nanodome SERS substrate. FIG. 14 shows the SERS spectra acquired on a gold nanodome substrate before and after trypsin cleavage for all three peptides. As seen in FIG. 14b, for CALNN(cnF)GGGGVRGNF (SEQ ID NO:11), there is a complete disappearance of the Phe peak at 1003 cm₋₁. FIG. 14c shows the same experiment for the CALNN(bzF)GGGGVRGNF (SEQ ID NO:12) substrate. Also here, a decrease in the 1003 cm⁻¹ peak is observed (note that part of its signal remains because benzoyl-phenylalanine also has a vibration at this exact frequency, as can be seen from its chemical structure).

This data demonstrates the possibility of interchanging aromatic amino-acids for acquiring specific signals. This will allow to design peptides with specific signals as substrates for specific proteases.

### Example 9. Waveguide-Based Detection and Parallelization of Measurements

An integrated photonics platform providing a lab-on-a-chip for detecting protease activity offers a number of advantages. Multiple waveguides can be integrated on a single chip for a parallel readout of multiple conditions. Furthermore the integration of various functions of traditional optical components on a single chip allows miniaturizing bulky analysis methods, thereby reducing both the size and cost of the required infrastructure. Finally, because the waveguide replaces the upright or inverted collection system (e.g., confocal microscope system), the surface of the integrated chip can be combined with microfluidics, which can greatly reduce sample consumption. These advantages are, for example, of great importance for single-cell analysis because a microscope-based, time-sequential readout is too slow for simultaneously monitoring a high number of single cells. Currently, the acquisition of one SERS spectrum requires a few seconds, while at least hundreds of cells need to be monitored in parallel to establish single-cell statistics.

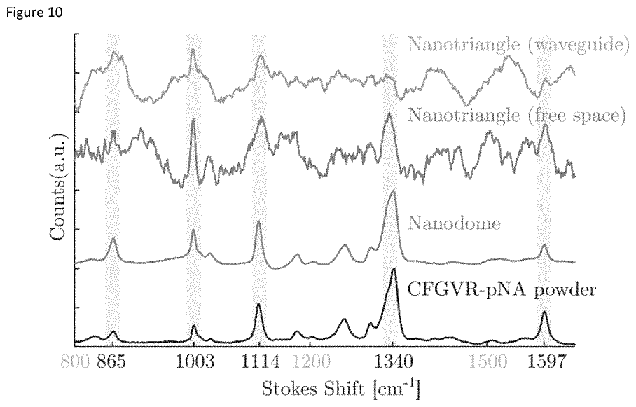

It was previously demonstrated how a silicon-nitride waveguide patterned with gold nanotriangles can be used for acquiring the SERS spectrum of a gold-bound peptide monolayer. These gold-nanotriangle-patterned waveguides were fabricated as described in Wuytens, Skirtach, & Baets (2017 Optics Express, 25: 12926-12934). FIG. 9 shows the nanotriangle-patterned waveguide and FIG. 10 shows the waveguide-detected SERS spectrum. Here, the short peptide CFGVR-pNA (a trypsin substrate; SEQ ID NO:15) is bound to the gold surface and the laser light was coupled into the waveguide. The gold nanostructures on top of the waveguide enhance the Raman signal, and part of the Stokes scattered light from the peptides couples back into the waveguide. Here, the collected SERS signal is imaged into an external Raman spectrometer with a CCD camera. However, the spectrometer and even the image sensor can also be partially integrated on the chip.

The waveguide-collected SERS spectrum is improved by reducing the background noise using on-chip filters and single-mode waveguides, as well as increasing the collected signal by improving the enhancement factor of the antennas.

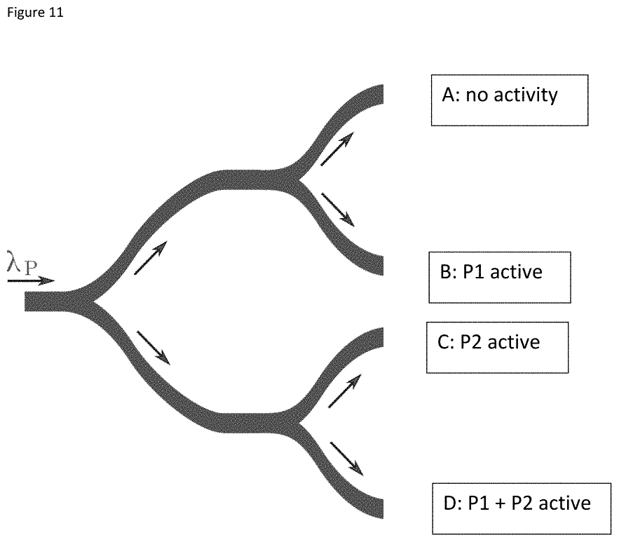

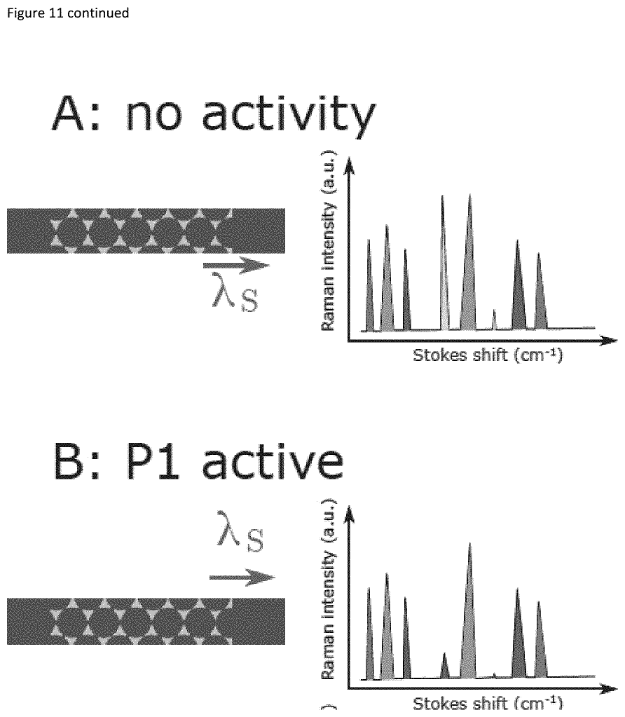

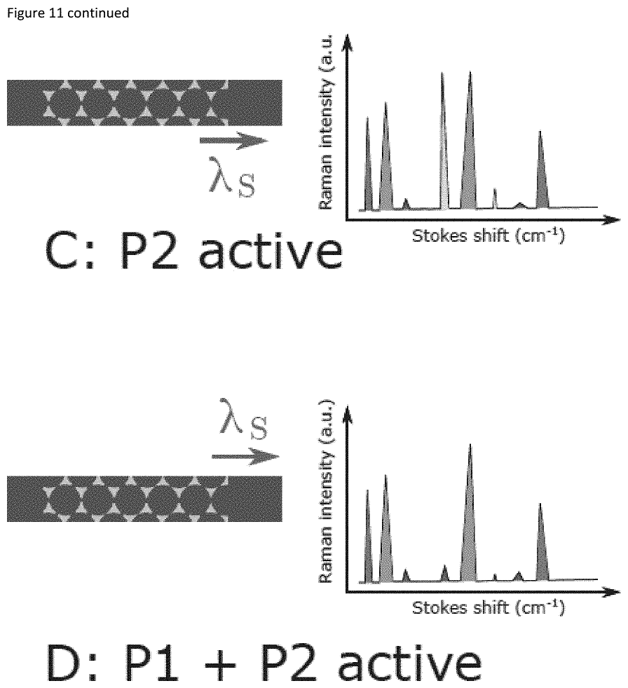

Apart from the waveguide-excitation and collection of the SERS spectra, the measurement principle is exactly the same. First, the chip is immersed with the peptide solution, after which a self-assembled monolayer is formed. Next, cleavage by a specific protease is monitored from the ratio of SERS bands measured via the waveguide. In a parallel chip, the incoming laser light is split in multiple (10-100) waveguide channels using Y-splitters. Each waveguide contains a stretch with gold nanostructures where the SERS signal is generated. An example of this structure is shown in FIG. 11. The collected SERS signal of each waveguide is imaged on one row of a 2D camera (CCD or CMOS), using an integrated spectrometer. Example applications are single-cell analysis, where the SERS pattern in each channel contains a single cell or protease inhibitor screening, where different conditions are applied to each channel using microfluidics.

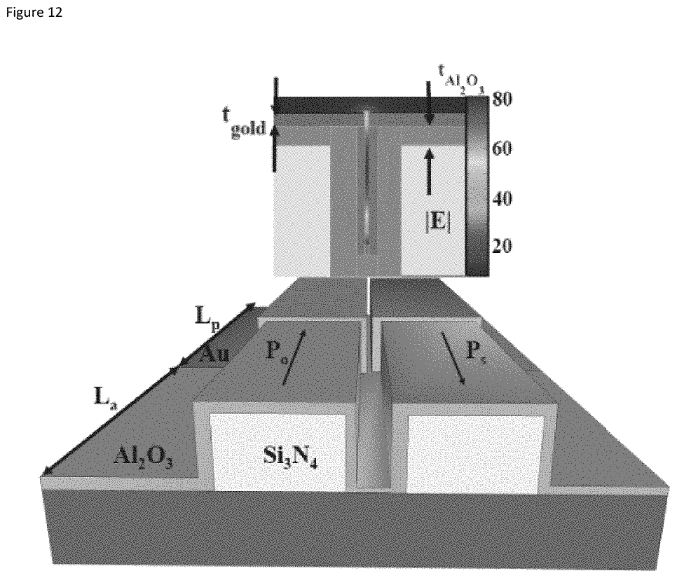

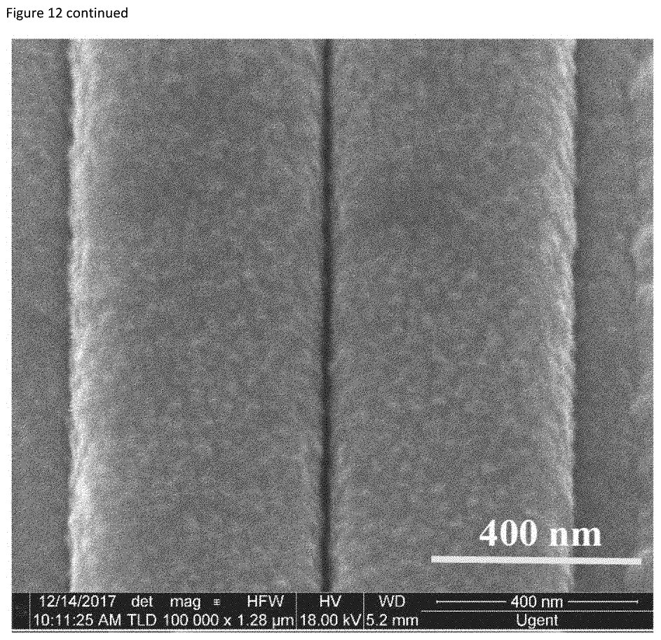

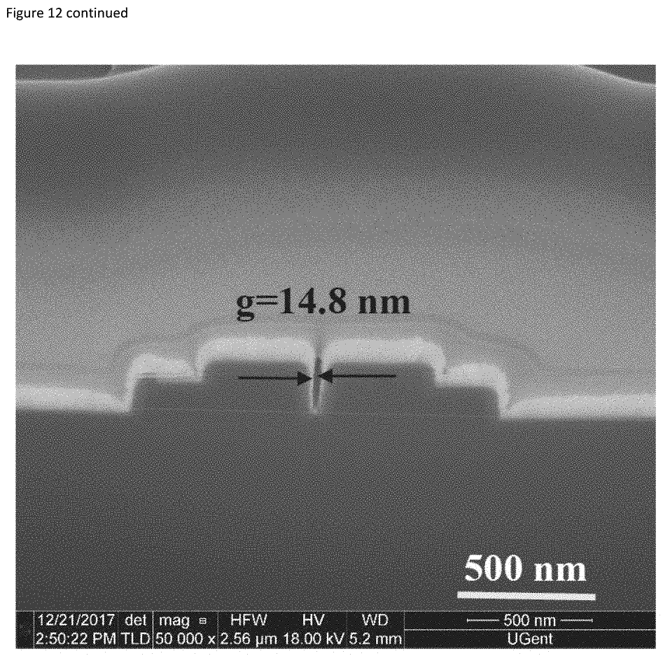

FIG. 12 shows another implementation of a waveguide-based SERS sensor. Here, a silicon nitride (Si₃N₄) slot waveguide guides the fundamental transverse-electric (TE) mode. The waveguide is narrowed down with an aluminum-oxide layer (Al₂O₃), deposited through atomic layer deposition (ALD). Locally, a gold layer is deposited in this narrowed slot. The result is a 15 nm wide slot containing a tightly confined propagating surface plasmon mode. This provides a strong field enhancement in the metal slot. The ALD-deposited layer in between allows to achieve a nm-accurate control on the slot dimension. An important asset of this fabrication procedure is that it can be scaled-up to wafer-scale processing. This offers perspective for high volume, low cost chip manufacturing.

Experimentally, it is found that 10⁻⁹ times the input power is converted to Stokes scattered power for a monolayer of para-nitrothiophenol. This is close to the enhancement achieved with the nanodome free-space SERS substrate, having a 10⁻⁸ conversion factor. Peptide signals are detected on these structures in a similar way as described above.

### Conclusions

SERS was successfully used to monitor protease-catalysed hydrolysis of a peptide by using the Raman fingerprint of aromatic amino acids. The peptide CALNNYGGGGVRGNF (SEQ ID NO:3) forms a stable monolayer of trypsin substrates on a gold-nanodome platform, whose plasmonic hotspots are accessible to proteases of 20-30 kDa. Real-time monitoring of trypsin activity on this gold-bound peptide shows immediate digestion within the first two minutes for a 8.6 nM concentration. It is further demonstrated that an analogous peptide sequence CALNNYGGGGNNESWH (SEQ ID NO:4) provides a good substrate for endoproteinase Glu-C and identify the SERS spectra of both substrates and their products, thereby taking a first step toward a label-free multiplexed measurement of protease activity. Further demonstrated is a quantification of the enzyme activity on gold-nanodome bound substrates for single and multiplexed protease activity. Importantly the disclosed method also works with non-natural aromatic amino acids as Raman active tags opening the possibility to multiplexing.

In summary, important advantages of the real-time detection of protease activity presented in this application over existing SERS-based techniques are the completely label-free measurements and the inherent control against ligand exchange. Furthermore a nanosphere-lithography based SERS platform is used, which offers controllable hotspots and is relatively easy and cheap to fabricate. In combination with microfluidics, SERS-based microchips can offer a promising platform for a sensitive, selective and multiplexed measurement of protease activity in various applications.

### Experimental Procedures

Materials

Para-nitrothiophenol (4-NTP, Sigma N27209), Endoproteinase Glu-C (Staphylococcus Aureus V-8 Protase, ThermoFisher 20195), Sequencing Grade Modified Trypsin (Promega V5111), Nα-Benzoyl-DL-arginine 4-nitroanilide hydrochloride (L-BAPNA, Sigma B4875), Ovomucoid (Type II-0) Trypsin inhibitor from chicken egg white (Sigma, T9253), Monodisperse Polystyrene Microbeads (microparticles GmbH, 448 nm), Dichloromethane (Sigma), Dimethylformamide (Sigma), Ammonium bicarbonate (Sigma), Acetonitrile (Sigma), Acetone (VWR), Isopropyl alcohol (VWR), Ethanol (Anhydrous, Sigma), Methanol (VWR), Sulfuric Acid (VWR), Hydrogen Peroxide (VWR).

Finite Difference Time Domain Simulations

Lumerical FDTD Solutions® was used for simulating the field profile in the plasmonic hotspots. A total-field scattered-field source (TFSF) is incident on a gold nanodome dimer in water on a Si¬3N4/Si substrate with 224 nm radius and 10 nm gap width with anti-symmetric, PML and symmetric boundaries along, respectively, X, Y and Z. All geometrical parameters were set to match the data from the SEM top view and cross-section images to the best of our ability. The refractive indices are set as follows: nSiN=2, nAu=Johnson and Christy [Johnson, P., & Christy, R. (1972). Optical Constants of Noble Metal. Physical Review B.], n¬water=1.33.

Peptide Synthesis

The peptides were synthesized using standard solid-phase Fmoc chemistry on a SyroI (biotage) instrument. The synthesis was started on 25 μmol preloaded Fmoc-His(Trt) or Fmoc-Phe wang resin, respectively, (novabiochem). The amino acids were coupled in a 4-fold excess using HOBT/HBTU activation. The peptides were cleaved with TFA containing phenol, triisipropylsilan and 5% H₂O for 3 hours. The peptides were precipitated with tributylmethyl ether and recovered by centrifugation at 2000 g. The ether washing/centrifugation step was repeated 3 times. The peptides were purified by a water/acetonitrile gradient elution on a RPC C18 column (Macherey-Nagel).

Peptide Assays

Peptides were first dissolved to 100 μg/μl in DMF and further diluted to 100 μg/ml in either a 10% acetonitrile/water mixture for labelling or a 50 mM ammonium bicarbonate buffer (pH 7.8) for bulk digestion experiments. Prior to labelling the chips with the peptides, they were cleaved into pieces of a few mm² and cleaned by sonication in acetone, rinsed with isopropyl alcohol and water and dried with under a stream of nitrogen. Next, remaining organic contaminants were removed in an 02 plasma, which also renders the surface hydrophilic (120 s, PVA-TEPLA GIGAbatch 310 M, 6000 sccm O2, 600 W, 750 mTorr). Immediately afterwards, the chips were immersed in separate wells of a polypropylene 96-well plate, using 100 μl of a 100 μg/ml peptide solution in 10% acetonitrile in water. After overnight incubation, the chips were rinsed excessively with deionized water. All assays were done in a freshly prepared 50-100 mM ammonium-bicarbonate buffer at 37° C. Both trypsin and endoproteinase Glu-C were first incubated at 37° C. for 15 min prior to addition to the peptide substrate, ensuring immediate maximal enzyme activity. The effectivity of the ovomucoid trypsin inhibitor for blocking trypsin digestion was tested in a 405 nm absorption assay (Tecan Infinite 200 PRO) on the commercially available L-BAPNA trypsin substrate. An excessive inhibitor concentration of 100 μg/ml is also used.

HPLC and Mass Spectrometry

After incubation for a specific time at 37° C., 750 μl of a solution with 100 μg/ml substrate and 3.3 μg/ml trypsin or endoproteinase Glu-C in 50-100 mM ammonium bicarbonate was injected in a C-18 reversed phase high-performance liquid chromatography column (Macherey-Nagel) and eluted by a water/acetonitrile gradient (Akta Purifier, GE). The molecular weight of separated fractions was determined using matrix-assisted laser desorption/ionisation time-of-flight mass spectrometry (Bruker MALDI-TOF).

Raman Microscopy

Spectra were acquired on a WITec Alpha 300 R+ confocal Raman microscope equipped with spectrometer using a 600 lpmm grating, a −70° C. cooled CCD camera (Andor iDus 401 BR-DD) and a 785 nm diode laser (Toptica, XTRA II). Raman spectra of the peptide powders were acquired using a Zeiss 100×/0.9 EC Epiplan NEOFLUAR; ∞/0 objective and a laser power of 100 mW, measured before the objective. SERS spectra were acquired through a Zeiss 63×/1.0 W-Plan Apochromat ∞/0 objective with 2 mW laser power. For real-time monitoring of enzymatic activity, the chip was placed in a metal petri dish filled with 1 ml 50 mM ammonium bicarbonate buffer (pH 7.8) inside a stage-top incubator at 37° C. (Okolab). Because of signal degradation upon laser illumination, each trace is the median spectrum of a spatially distributed map of 10×10 pixels in a 20×20 μm area with an integration time of 1 s on each point. In the real-time assay, every next trace was mapped on a different location on the SERS platform. A limited amount of inhomogeneity across the gold nanodome area accounts for the variation in 11003/1829-860 visible in FIG. 7(c).

SERS Data Processing

Starting from the 10×10 individual spectra, WITec Project Four® was used for removing cosmic rays. Next, the data was exported to Matlab® and aberrant spectra were rejected using a variance-based filter, after which the background of each individual spectrum is subtracted using a high-pass filter. All SERS spectra plotted in this paper are the median spectra of these data. 11003 and 1829-860 were calculated by integrating the peaks at their respective positions and subtracting the background with a linear fit for the individual spectra. The boxplots in FIG. 6 are based on these individual peak intensities.

SERS Enhancement Factor Calculations

After cleaning, chips were immersed overnight in a 1 mM solution of 4-NTP in ethanol and rinsed excessively in ethanol. The SERS36 substrate enhancement factor is calculated from the ratio between the Raman signal per molecule in a bulk solution (p=100 mM in ethanol, 10×/0.3 Nikon PlanFluor objective, 100 mW, 0.13 s) and the SERS signal per adsorbed molecule on the nanodome surface (10×/0.3 Nikon PlanFluor objective, 0.1 mW, 0.13 s):

SSEF=(I_SERS N_Vol)/(I_Raman N_Surf)=I_SERS/I_Raman (H_eff φ/(μ_Au μ_NTP A_m)

For this calculation, an effective height (Heff) of the confocal volume of 160 μm was used, a factor 1.6 for the ratio of nanodome surface surface area over projected surface area (μAuAm) and a loading density (μNTP) of 4.4×10⁶ molecules/μm² (ref 42).

Nanodome fabrication 4″ wafers patterned with nanodome substrates were fabricated by optimizing an earlier published protocol 21, as schematically shown in FIG. 2(a). First, a 200 nm layer of PECVD Si3N4 was deposited on top of a 4″ (100) Si wafer (Advanced Vacuum Vision 310-PECVD). Next, the wafer was cleaned and made hydrophilic by 20 min of 02 plasma (PVA-TEPLA GIGAbatch 310 M, 6000 sccm O2, 600 W, 750 mTorr) and stored in DI water. Prior to spin coating, the wafer was flash-dried under a stream of nitrogen, followed by spin-coating 760 μl of a 5 w/v % in 2/1 methanol/water mixture of 448 nm diameter polystyrene beads on top of the wafer. The exact spin speed and acceleration depend on environmental parameters such as humidity and temperature. Typically, a two-step process was used, first generating the hexagonally-packed monolayer by spinning at 900 rpm and an acceleration of 800 rpm/s for 100 s, followed by a faster spinning at 7000 rpm for 40 s to remove excess beads and solvent and finally again flash-drying the wafer with nitrogen. The HCP-layer of polystyrene beads was then transferred into the underlying Si3N4 using a two-step reactive ion etch. First, the diameter of the beads was reduced in an 02 plasma (Advanced Vacuum Vision 320-ME, 50 sccm 02, 75 W, 100 mTorr, 40-70 s), followed by a ¬CF4/H2 Si₃N₄ etch using an optimized recipe for anisotropic etching (80 sccm CF₄, 3 sccm H₂, 210 W, 20 mTorr, 70-100 s). These two steps, respectively, determine the width and height of the gap in between the nanodomes, the two most important parameters for tuning the plasmonic resonance, enhancement factor and hot-spot accessibility. Next, the beads were lifted off in dichloromethane and the wafers were cleaned in a piranha solution (MS0441202, 3/1, 15 minutes at 80° C.) before sputtering of a 2 nm thick Ti adhesion layer and a 130 nm thick Au layer (Alcatel SCM600, 10-2 mbar, 1 kW, rotating substrates). The chips were characterized through scanning electron microscopy on a FEI Nova 600 Nanolab Dual-Beam. FIB system, using a voltage of 18 kV and a through the lens (TLD) detection.

### REFERENCES

1 M. Drag and G. S. Salvesen, Nat. Rev. Drug Discov., 2010, 9, 690-701.
2 B. Turk, Nat. Rev. Drug Discov., 2006, 5, 785-799.
3 K. Y. Choi, M. Swierczewska, S. Lee and X. Chen, Theranostics, 2012, 2, 156-179.
4 M. Wu and A. K. Sing, Curr. Opin. Biotechnol., 2012, 23, 83-88.
5 T. Liu, Y. Yamaguchi, Y. Shirasaki, K. Shikada, M. Yamagishi, K. Hoshino, T. Kaisho, K. Takemoto, T. Suzuki, E. Kuranaga, O. Ohara and M. Miura, Cell Rep., 2014, 8, 974-982.
6 D. Maysinger and E. Hutter, Nanomedicine, 2015, 10, 483-501.
7 Y.-P. Kim and H. S. Kim, ChemBioChem, 2016, 17, 275-282.
8 K. Welser, R. Adsley, B. M. Moore, W. C. Chan, J. W. Aylott and C. Chan, Analyst, 2011, 136, 29-41.
9 Y. Lai, S. Sun, T. He, S. Schlücker and Y. Wang, RSC Adv., 2015, 5, 13762-13767.
10 G. L. Liu, Y. T. Rosa-Bauza, C. M. Salisbury, C. Craik, J. a Ellman, F. F. Chen and L. P. Lee, J. Nanosci. Nanotechnol., 2007, 7, 2323-2330.
11 C. Sun, K. H. Su, J. Valentine, Y. T. Rosa-Bauza, J. A. Ellman, 0. Elboudwarej, B. Mukherjee, C. S. Craik, M. A. Shuman, F. F. Chen and X. Zhang, ACS Nano, 2010, 4, 978-984.
12 C. Fu, W. Xu, G. Chen and S. Xu, Analyst, 2013, 138, 6282-6.
13 L. Chen, X. Fu and J. Li, Nanoscale, 2013, 5, 5905-11.
14 Z. Wu, Y. Liu, Y. Liu, H. Xiao, A. Shen, X. Zhou and J. Hu, Biosens. Bioelectron., 2015, 65, 375-381.
15 A. Ingram, L. Byers, K. Faulds, B. D. Moore and D. Graham, J. Am. Chem. Soc., 2008, 130, 11846-11847.
16 I. A. Larmour, K. Faulds and D. Graham, Chem. Sci., 2010, 1, 151-160.
17 B. D. Moore, L. Stevenson, A. Watt, S. Flitsch, N. J. Turner, C. Cassidy and D. Graham, Nat. Biotechnol., 2004, 22, 1133-8.
18 F. Wei, D. Zhang, N. J. Halas and J. D. Hartgerink, J. Phys. Chem. B, 2008, 112, 9158-9164.
19 E. Jorgenson and A. Ianoul, J. Phys. Chem. B, 2017, 121, 967-974.
20 R. Lévy, N. T. K. Thanh, R. Christopher Doty, I. Hussain, R. J. Nichols, D. J. Schiffrin, M. Brust and D. G. Fernig, J. Am. Chem. Soc., 2004, 126, 10076-10084.
21 P. C. Wuytens, A. Z. Subramanian, W. H. De Vos, A. G. Skirtach and R. Baets, Analyst, 2015, 140, 8080-8087.
22 V. See, P. Free, Y. Cesbron, P. Nativo, U. Shaheen, D. J. Rigden, D. G. Spiller, D. G. Fernig, M. R. H. White, I. A. Prior, M. Brust, B. Lounis and R. Levy, ACS Nano, 2009, 3, 2461-2468.
23 P. Free, C. P. Shaw and R. Levy, Chem. Commun., 2009, 33, 5009-5011.
24 S. S. Masango, R. A. Hackler, N. Large, A. I. Henry, M. O. McAnally, G. C. Schatz, P. C. Stair and R. P. Van Duyne, Nano Lett., 2016, 16, 4251-4259.
25 M. Gellner, K. Kompe and S. Schlücker, Anal. Bioanal. Chem., 2009, 394, 1389-1844.
26 E. Colangelo, Q. Chen, A. M. Davidson, D. Paramelle, M. B. Sullivan, M. Volk and R. Levy, Langmuir, 2017, 33, 438-449.
27 H. Häkkinen, Nat. Chem., 2012, 4, 443-455.
28 L. Duchesne, D. Gentili, M. Comes-Franchini and D. G. Fernig, Langmuir, 2008, 24, 13572-13580.
29 E. Vandermarliere, M. Mueller and L. Martens, Mass Spectrom. Rev., 2013, 32, 453-465.
30 M. Pozsgay, G. Szabó, S. Bajusz, R. Simonsson, R. Gaspar and P. Elödi, Eur. J. Biochem., 1981, 115, 497-502.
31 K. Breddam and M. Meldal, Eur. J. Biochem., 1992, 206, 103-107.
32 H. P. Erickson, Biol. Proced. Online, 2009, 11, 32-51.
33 L. Bayne, R. V. Ulijn and P. J. Halling, Chem. Soc. Rev., 2013, 42, 9000-9010.
34 E. Le Ru and P. Etchegoin, Principles of Surfance Enhanced Raman Spectroscopy and related plasmonic effets, Elsevier, 2009, vol. 1.
35 A. Yashchenok, A. Masic, D. Gorin, B. S. Shim, N. A. Kotov, P. Fratzl, H. Mohwald and A. Skirtach, Small, 2013, 9, 351-6.
36 E. C. Le Ru, M. Meyer and P. G. Etchegoin, J. Phys. Chem. C, 2007, 111, 13794-13803.
37 J. De Gelder, K. De Gussem, P. Vandenabeele and L. Moens, J. Raman Spectrosc., 2007, 38, 1133-1147.
G. Zhu, X. Zhu, Q. Fan and X. Wan, Spectrochim. Acta—Part A Mol. Biomol. Spectrosc., 2011, 78, 1187-1195.
39 J. Zhang, Q. Chi, J. U. Nielsen, E. P. Friis, J. E. T. Andersen and J. Ulstrup, Langmuir, 2000, 16, 7229-7237.
40 S. Nayak, W. Yeo and M. Mrksich, Langmuir, 2007, 172, 5578-5583.
41 O. A. Gutiérrez, M. Chavez and E. Lissi, Anal. Chem., 2004, 76, 2664-2668.
42 L. Baia, M. Baia, J. Popp and S. Astilean, J. Phys. Chem. B, 2006, 110, 23982-23986.
43 R. Derda, D. J. Wherritt and L. L. Kiessling, Langmuir, 2007, 23, 11164-11167.
44 Movasaghi, Z., Rehman, S., & Rehman, I. U. Applied Spectroscopy Reviews, 2007, 42: 493-541.

1. A peptide with a maximum length of 35 amino acids, the peptide comprising a protease recognition sequence and at least two (2) Raman active tags,
wherein the at least two (2) Raman active tags comprise at least one (1) Raman active tag N-terminally from the protease recognition sequence and at least one (1) Raman active tag C-terminally from the protease recognition sequence.

2. The peptide according to claim 1, wherein the Raman active tag is a tag for surface enhanced Raman scattering (SERS) detection.

3. The peptide according to claim 1, wherein the Raman active tags are natural and/or non-natural aromatic amino acids.

4. The peptide according to claim 1, wherein at least one (1) of the at least two (2) Raman active tags is a non-natural aromatic amino acid or a Raman active tag, which is not part of the peptide under natural conditions.

5. The peptide according to claim 1, comprising SEQ ID NO:1.

6. The peptide according to claim 1, comprising a peptide having at least 90% homology to SEQ ID NO:2: CALNNXGGGG, wherein X can be any natural or non-natural aromatic amino acid.

7. A carrier having a molecule attached to it, the molecule comprising an enzyme recognition sequence and at least 2 least two (2) Raman active tags, wherein the molecule comprises at least one (1) Raman active tag before the enzyme recognition sequence and at least one (1) Raman active tag behind the enzyme recognition sequence.

8. A carrier having n different molecules attached to it, wherein then different molecules differ from each other by comprising a different enzyme recognition sequence, wherein every molecule comprises at least one Raman active tag before and at least one Raman active tag behind the enzyme recognition sequence, wherein the ratios of the Raman intensity of the at least one Raman tag behind the enzyme recognition sequence and the Raman intensity of the at least one Raman tag before the enzyme recognition sequence or vice versa is specific for every molecule comprising a different enzyme recognition sequence, and wherein n is an integer between 2 and 100.

9. The carrier according to claim 7, wherein the molecule is a polynucleotide and wherein the enzyme is a nuclease.

10. The carrier according to claim 7, wherein the molecule is a peptide and wherein the enzyme is a protease.

11. The carrier according to claim 7, wherein the molecule comprises a peptide having a maximum length of 35 amino acids,
wherein the peptide comprises a protease recognition sequence and at least two (2) Raman active tags, the at least two (2) Raman active tags comprising at least one (1) Raman active tag N-terminally from the protease recognition sequence and at least one (1) Raman active tag C-terminally from the protease recognition sequence.

12. The carrier according to claim 8, wherein the carrier comprises a material selected from the group consisting of gold, silver, silicon, silicon nitride, silicon dioxide, quartz, polystyrene, silica and dextran.

13. (canceled)

14. A method of detecting or quantifying the presence, amount, and/or activity of at least one enzyme in a biological sample, the method comprising:
utilizing the peptide according to claim 1 to detect or quantify the presence, amount or activity of at least one enzyme in a in the biological sample.

15. A method of detecting or quantifying, in parallel, the presence, amount, or activity of at least two enzymes in one biological sample, the method comprising:
utilizing the carrier according to claim 8 to detect or quantify in parallel the presence, amount or activity of at least two enzymes in one biological sample.

16. The method according to claim 14, the method comprising:
a. contacting the carrier with the sample, wherein a molecule attached to the carrier comprises a recognition sequence for the enzyme; and
b. measuring the Raman ratio before and after contacting the sample with the carrier, wherein the Raman ratio is the ratio between the Raman intensity of the one or more Raman active tags behind the enzyme recognition sequence of the molecule and the Raman intensity of the one or more Raman active tags before the enzyme recognition sequence of the molecule or vice versa;
wherein a change in Raman ratio before and after contacting the sample with the carrier gives a metric for the presence, amount or activity of the enzyme in the sample.

17. The method according to claim 15, the method comprising:
a. contacting the carrier with the sample, wherein the n different molecules attached to the carrier comprise the recognition sequences for then different enzymes; and
b. measuring the Raman ratio before and after contacting the sample with the carrier, wherein the Raman ratio is the ratio between the Raman intensity of the one or more Raman active tags behind the enzyme recognition sequence of then different molecules and the Raman intensity of the one or more Raman active tags before the enzyme recognition sequence of then different molecules or vice versa;
wherein for each different enzyme and for the Raman active tags of a molecule comprising the recognition sequence of the enzyme, the change in Raman ratio before and after contacting the sample with the carrier gives a metric for the presence, amount or activity of then different enzymes in the sample.

18. The method according to claim 17, wherein the enzyme is a protease or a nuclease.

19. The method according to claim 17, wherein the Raman scattering is excited and collected by one or more integrated optical waveguides.

20. The method according to claim 19, wherein the one or more integrated optical waveguides are integrated dielectric optical waveguides and patterned with gold, silver, copper or aluminum nanostructures.

21. A method of screening for a compound with protease or nuclease activity, the method comprising:
a. contacting the carrier of claim 7 with at least one test compound;
b. measuring the Raman intensities of the at least two Raman active tags of the one or more different molecules attached to the carrier before and after contacting the test compound with the carrier; and
c. identifying the test compound as a compound with protease or nuclease activity, if at least one Raman ratio decreases with at least 25% after contacting the carrier with the test compound, wherein the Raman ratio is the ratio between the Raman intensity of the one or more Raman active tags behind an enzyme recognition sequence of a molecule attached to the carrier and the Raman intensity of the one or more Raman active tags before the enzyme recognition sequence.

22. A method of screening for a compound that inhibits the activity of a protease or nuclease, the method comprising:
a. contacting the carrier of claim 7 with at least one test compound;
b. measuring the Raman intensities of the at least two Raman active tags of the one or more molecules attached to the carrier before and after contacting the test compound with the carrier in the presence of the protease or nuclease, wherein the one or more molecules comprises a recognition sequence for the protease or nuclease; and
c. identifying the test compound as a compound that inhibits activity of the protease or nuclease, if the Raman ratio in the presence of the test compound is at least 25% higher than the Raman ratio in the absence of the test compound, or, if the Raman ratio in the absence of the test compound is at least 25% lower that the Raman ratio in the presence of the test compound, wherein the Raman ratios are the ratios between the Raman intensity of the one or more Raman active tags behind an enzyme recognition sequence of a molecule attached to the carrier and the Raman intensity of the one or more Raman active tags before the enzyme recognition sequence.
